SEQUENCE LISTING (19 sequences)

SEQ ID NO: 1 - DNA, 21 bp, Artificial Sequence
  Primer
cctaaggttaagtcgccctcg

SEQ ID NO: 2 - DNA, 21 bp, Artificial Sequence
  Primer
cctcagcagaactcactaaat

SEQ ID NO: 3 - DNA, 21 bp, Artificial Sequence
  Primer
aagctacatacccagccctcc

SEQ ID NO: 4 - DNA, 21 bp, Artificial Sequence
  Primer
tggaaactgttacgcttctca

SEQ ID NO: 5 - DNA, 17 bp, Artificial Sequence
  Primer
gtctctagccctacccg

SEQ ID NO: 6 - DNA, 38 bp, Artificial Sequence
  Primer
gtgagctttcttatcagtctgccctcattgtcggtttt

SEQ ID NO: 7 - DNA, 38 bp, Artificial Sequence
  Primer
gtgagctttcttatcagtctgaactacttgtcggtttt

SEQ ID NO: 8 - DNA, 19 bp, Artificial Sequence
  Primer
ggactacatccagaccgac

SEQ ID NO: 9 - DNA, 18 bp, Artificial Sequence
  Primer
tgggactccgtgaggaac

SEQ ID NO: 10 - DNA, 19 bp, Artificial Sequence
  Primer
tctggaggtcccctggtta

SEQ ID NO: 11 - DNA, 19 bp, Artificial Sequence
  Primer
agcatcatcaccccaatgt

SEQ ID NO: 12 - DNA, 20 bp, Artificial Sequence
  Primer
gagggctggtcacatgaaga

SEQ ID NO: 13 - DNA, 18 bp, Artificial Sequence
  Primer
gctccgctaatttccagt

SEQ ID NO: 14 - DNA, 17 bp, Artificial Sequence
  Primer
gtcagcaccaaacagcg

SEQ ID NO: 15 - DNA, 20 bp, Artificial Sequence
  Primer
ggagattccatcagtcaccc

SEQ ID NO: 16 - DNA, 23 bp, Artificial Sequence
  Primer
ctgacaaggcttttttcttcaca

SEQ ID NO: 17 - DNA, 17 bp, Artificial Sequence
  Primer
ccagacgggacaggttt

SEQ ID NO: 18 - DNA, 22 bp, Artificial Sequence
  Primer
aactccatcatgaagtgtgacg

SEQ ID NO: 19 - DNA, 20 bp, Artificial Sequence
  Primer
gatccacatctgctggaagg

(19) United States
(12) Patent Application Publication
(10) Pub. No.: US 2014/0113288 A1
(43) Pub. Date: Apr. 24, 2014

(54) METHODS OF IDENTIFYING RISK OF PREECLAMPSIA AND PREGNANCY-RELATED DISORDERS

(71) Applicant: Academia Sinica, Taipei (TW)

(72) Inventors: Hungwen Chen, Taipei (TW); Liang-Jie Wang, Taipei (TW)

(73) Assignee: Academia Sinica, Taipei (TW)

(21) Appl. No.: 14/057,038

(22) Filed: Oct. 18, 2013

Related U.S. Application Data
(60) Provisional application No. 61/716,754, filed on Oct. 22, 2012.

(51) Int. Cl. C12Q 1/68; G01N 33/68

(52) U.S. Cl. CPC C12Q 1/6883; G01N 33/689
USPC 435/6.11; 436/501; 435/7.92; 435/6.12

(57) ABSTRACT

Provided herein are methods of diagnosing a pregnancy disorder (e.g., preeclampsia or related pregnancy disorders) in a subject based on the level of High Temperature Requirement A4 (HtrA4). Also provided herein are diagnostic agents and devices for use in such diagnostic methods.

### RELATED APPLICATION

This application claims the benefit of U.S. provisional application No. 61/716,754, filed Oct. 22, 2012 under 35 U.S.C. §119, the entire content of which is herein incorporated by reference.

### BACKGROUND OF THE INVENTION

Human placentation proceeds fast after embryo implantation such that different classes of specialized trophoblast cells have evolved to establish blood circulation for nutrient, gas, and waste exchange between mother and fetus. The mononuclear cytotrophoblasts in chorionic villi proliferate and differentiate through cell-cell fusion into a multinucleated syncytiotrophoblast layer, which is in direct contact with maternal blood to mediate the abovementioned exchanges and produce hormones and growth factors for pregnancy maintenance. On the other hand, cytotrophoblasts in the chorionic villi that anchor to uterine decidua proliferate into cell columns from where some cytotrophoblasts migrate and invade deeper layers of decidua. The migratory and invasive cytotrophoblasts are termed interstitial extravillous trophoblasts (EVTs), which may further invade the uterine myometrium and replace the endothelial cells of spiral arteries. This phenomenon, called spiral artery remodeling, is essential for sufficient blood flow into intervillous spaces of the placenta as remodeled arteries become dilated and non-vasoactive. Insufficient spiral artery remodeling due to shallow trophoblast invasion may result in placental hypoxia and pregnancy complications such as preeclampsia and intra-uterine growth retardation with clinical features of gestational hypertension, proteinuria, and failure of optimal fetal growth (6).

GCM1, also known as GCMa, is a transcription factor critical for placental development (2, 33). GCM1 regulates expression of syncytin-1 and -2 fusogenic proteins for syncytiotrophoblast differentiation and placental growth factor (PGF) for placental vasculogenesis (9, 19, 22, 37). The syncytin-1 and -2 proteins are encoded by envelope genes of human endogenous retroviruses, HERV-W and HERV-FRD, respectively (4, 5, 28). The syncytin polypeptide is post-translationally cleaved into surface (SU) and transmembrane (TM) subunits, which mediate receptor recognition and membrane fusion, respectively. GCM1 activity is inhibited under hypoxia, where GSK-3β mediates Ser322 phosphorylation leading to GCM1 ubiquitination and degradation (12). This may underscore decreased GCM1 and PGF expression in the hypoxic preeclamptic placentas. By contrast, cAMP signaling stimulates GCM1 gene transcription (19) and enhances GCM1 stability by facilitating dual-specificity phosphatase 23-mediated Ser322 dephosphorylation and CREB-binding protein-mediated GCM1 acetylation, providing the underpinnings of the long-known stimulation of trophoblastic fusion by cAMP (8, 23).

### SUMMARY OF THE INVENTION

The present disclosure is based on the unexpected discovery that High Temperature Requirement A4 (HtrA4) is a novel target gene of GCM1 and the levels of HtrA4 in pregnant women is associated with pregnancy disorders such as preeclampsia. As such, HtrA4 can serve as a reliable biomarker for diagnosing pregnancy disorders, monitoring the development of pregnancy, and/or assessing the efficacy of a treatment of the pregnancy disorder.

Accordingly, in some aspects, provided herein are methods of diagnosing (e.g., identifying a subject having or assessing the risk for) a pregnancy disorder in a subject, e.g., preeclampsia or intrauterine growth retardation (IUGR). The methods comprises providing a biological sample of a pregnant subject (e.g., a human patient), measuring a level of High Temperature Requirement A4 (HtrA4) in the biological sample, and identifying the pregnant subject as having or at risk for a pregnancy disorder based on the level of HtrA4. If the level of HtrA4 deviates from (e.g., is lower than or higher than) a reference value, the pregnant subject is identified as a subject having or at risk for a pregnancy disorder. In some examples, the biological sample, which can be a serum sample or a plasma sample, can be obtained from the pregnant subject at 10 weeks to 38 weeks of pregnancy. In some examples, the level of HtrA4 is a level of HtrA4 protein and optionally, the protein level of HtrA4 is measured in an assay that involves an anti-HtrA4 antibody, e.g., an enzyme-linked immunosorbent assay (ELISA) or an immunoblotting assay. In other examples, the level of HtrA4 is a level of HtrA4 gene transcript and optionally, the gene transcript level of HtrA4 is measured in an assay that involves an oligonucleotide complementary to the HtrA4 gene transcript or a fragment thereof, e.g., a hybridization assay or a real-time PCR assay.

The method described herein can further comprise monitoring progress of pregnancy disorder in the pregnant subject if the subject is identified as having or at risk for the pregnancy disorder, for example, preeclampsia or IUGR. Alternatively or in addition, the method further comprises treating the pregnant subject to alleviate a symptom associated with the pregnancy disorder. Such treatments include, but are not limited to, smoking cessation, antihypertensive therapy, magnesium sulfate treatment, dietary and nutritional factor treatment, aspirin supplementation treatment, exercise, induction of paternal tolerance, and administration of immune factors.

In other aspects, the present disclosure provides methods for monitoring development (e.g., on-set or progress) of a pregnancy disorder (e.g., preeclampsia or IUGR). The method comprises providing a first biological sample of a pregnant subject (e.g., a human patient) at a first gestation stage; measuring a first level of HtrA4 in the first biological sample; providing a second biological sample of the pregnant subject at a second gestation stage subsequent to the first gestation stage; measuring a second level of HtrA4 in the second biological sample; and assessing development of a pregnancy disorder in the pregnant subject based on the change of the levels of HtrA4 in the first and second biological samples. If the second level of HtrA4 deviates from the first level of HtrA4, it indicates that the pregnancy disorder progresses in the pregnant subject or the pregnant subject has developed or is at risk for developing the pregnancy disorder.

In some embodiments, the first biological sample, the second biological sample, or both are serum samples or plasma samples. In some embodiments, the first biological sample is obtained from the pregnant subject at 10 weeks to 38 weeks of pregnancy. The second biological sample can be obtained from the pregnant subject at least two weeks after obtaining the first biological sample.

In some embodiments, the level of HtrA4 is a level of HtrA4 protein and optionally, the protein level of HtrA4 is measured in an assay that involves an anti-HtrA4 antibody, e.g., an enzyme-linked immunosorbent assay (ELISA) or an immunoblotting assay. In other embodiments, the level of HtrA4 is a level of HtrA4 gene transcript and optionally, the gene transcript level of HtrA4 is measured in an assay that involves an oligonucleotide complementary to the HtrA4 gene transcript or a fragment thereof, e.g., a hybridization assay or a real-time PCR assay.

The method described herein can further comprise monitoring progress of pregnancy disorder in the pregnant subject if the subject is identified as having or at risk for the pregnancy disorder, for example, preeclampsia or IUGR. Alternatively or in addition, the method further comprises treating the pregnant subject to alleviate a symptom associated with the pregnancy disorder. Such treatments include, but are not limited to, smoking cessation, antihypertensive therapy, magnesium sulfate treatment, dietary and nutritional factor treatment, aspirin supplementation treatment, exercise, induction of paternal tolerance, and administration of immune factors.

In some embodiments, the first and second biological samples are obtained before and after a treatment to alleviate a symptom of the pregnancy disorder (e.g., preeclampsia). In other embodiments, the first and second biological samples are obtained during a course of a treatment to alleviate a symptom of the pregnancy disorder. When needed, the method further comprises changing the treatment for alleviating the symptom of the pregnancy disorder.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The drawings are first described.

FIG. 1. demonstrates GCM1 regulation of placental cell invasion. (A) Expression of GCM1 in JAR and BeWo cells. JAR and BeWo cells were subjected to immunoblotting with GCM1 and β-actin Abs. (B) Stimulation of JAR cell invasion by GCM1. Mock (pCDH) and HA-GCM 1-expressing (pCDH-HA-GCM 1) JAR cells were plated into Matrigel-coated chambers for invasion analysis. Representative pictures of cells that migrated to and invaded the lower surface of filters are provided. Mean values and the SD obtained from three independent experiments are presented. (C) GCM1 knockdown suppresses BeWo cell invasion. BeWo cells stably expressing scramble or GCM1 shRNA were subjected to invasion analysis as described in (A). (D) Identification of GCM1 target genes. BeWo31 cells were treated with 50 μM forskolin for 24 h, followed by ChIP-chip analysis as described in Materials and Methods. HtrA4 was identified as a candidate GCM1 target gene with strong hybridization signals (gray versus black) in the region upstream of the exon1 of HtrA4 gene (right). PGF, a known GCM1 target gene, was also identified in the analysis (left).

FIG. 2. demonstrates regulation of HtrA4 expression by GCM1. (A) GCM1 knockdown decreases HtrA4 expression. BeWo cells stably expressing scramble or GCM1 shRNA were subjected to immunoblotting with GCM1, HtrA4, and β-actin Abs, respectively. In a separate experiment, cells were immunostained with HtrA4 antibody (Ab) (green) and nuclei were stained by 4′,6-diamidino-2-phenylindole (DAPI) (blue), followed by confocal microscopy analysis. Note that HtrA4 signals in the cytoplasm were decreased in the GCM1-knockdown BeWo cells. (B) Expression of HtrA4, but not other HtrA family members, is decreased by GCM1 knockdown. BeWo cells stably expressing scramble or GCM1 shRNA were harvested for quantitative PCR analysis of the transcript levels of HtrA family members. Mean values and the SD obtained from three independent experiments are presented. (C) Overexpression of GCM1 stimulates HtrA4 expression. Mock and HA-GCM1-expressing JAR cells were harvested for immunoblotting and quantitative PCR analysis of HtrA4 protein and transcript levels, respectively. (D) Regulation of HtrA4 promoter activity by GCM1. Schematic representation of HtrA4 promoter region with the wild-type and mutant GCM1-binding site (GBS) is provided (upper). 293T cells were transfected with pHtrA4-1 kb or pHtrA4-1 kbGBSmt plus or minus pHA-GCM1 expression plasmid (left). BeWo cells expressing scramble or GCM1 shRNA were transfected with pHtrA4-1 kb or pHtrA4-1 kbGBSmt (right). 48 hours post-transfection, cells were harvested for luciferase assays. Mean values and the SD obtained from three independent experiments are presented. (E) Interaction of GCM1 and the GBS in HtrA4 promoter. Recombinant GCM1-FLAG was incubated with radiolabeled HtrA4-GBS or HtrA4-GBSmt probes in the presence of GCM1 or syncytin-2 (Syn2) Ab in EMSA. The asterisk and arrow indicate the GCM1-FLAG-DNA complex and its supershifted complex, respectively. Association of GCM1 and HtrA4-GBS in BeWo cells was analyzed by ChIP using normal rabbit serum (NS) or GCM 1 Ab for immunoprecipitation and designated primer pairs for PCR.

FIG. 3. demonstrates coexpression of GCM1 and HtrA4 in the interstitial EVTs of human placenta. (A) Immunostaining of GCM1 and HtrA4 in term placenta. Term placental tissues sections were subjected to immunohistochemistry using GCM1 (a and d), HtrA4 (b and e), and CK7 (c and f) Abs and further counterstained by hematoxylin. Note that expression of GCM 1 corresponded with that of HtrA4 in the CK7-positive EVTs in the consecutive sections of basal plate (a-c). The insets in panels d-f show sections immunostained with normal rabbit, guinea pig, and mouse serum, respectively. Bar, 100 μm. (B) Purification of EVTs. Primary trophoblast cells prepared from term placenta were subjected to flow cytometry analysis using HLA-G Ab as described in Materials and Methods. (C) Expression of HtrA4 and GCM1 in EVTs. HLA-G-positive EVTs were subjected to single cell RT-PCR as described in Materials and Methods. Amplification curves of real-time PCR reactions of increasing input cell numbers are presented. Of note, neither GCM1 nor HtrA4 transcript was detected in the samples without reverse transcriptase (RT) and the mock reactions without input sample (NG). The PCR products were analyzed by agarose gel electrophoresis (upper panel). MW, molecular weight marker. (D) Colocalization of GCM1 and HtrA4 in EVTs. HLA-G-positive EVTs were immunostained with GCM1, HtrA4, and CK7 Abs, followed by confocal microscopy analysis. In a separate experiment, cells were transduced with lentivirus harboring scramble or GCM1 shRNA and subsequently harvested for quantitative PCR analysis of the HtrA4 and GCM1 transcripts. Mean values and the SD obtained from three independent experiments are presented.

FIG. 4. demonstrates HtrA4 regulation of placental cell invasion. (A) Overexpression of HtrA4 stimulates placental cell invasion. Mock and HtrA4-HA-expressing JAR cells were plated into Matrigel-coated chambers for invasion analysis. Expression of HtrA4-HA was analyzed by immunoblotting with HA mAb (left). (B) HtrA4 knockdown suppresses placental cell invasion. BeWo cells stably expressing scramble or HtrA4 shRNA were subjected to invasion analysis as described in (A). Knockdown of HtrA4 expression in BeWo cells were analyzed by immunoblotting with HtrA4 Ab (left). (C) Proteolytic cleavage of fibronectin by HtrA4. Purified HtrA4-FLAG and HtrA4mt-FLAG proteins from culture medium of 293T cells transfected with pHtrA4-FLAG and pHtrA4mt-FLAG, respectively, were analyzed by immunoblotting with FLAG mAb (left). Fibronectin was incubated with increasing amounts of HtrA4-FLAG or HtrA4mt-FLAG at 37° C. for 16 h. The reaction mixture was then subjected to immunoblotting with HtrA4 or fibronectin Ab. Of note, partial cleavage of HtrA4-FLAG, but not HtrA4mt-FLAG, in the reaction was detected (lower).

FIG. 5. demonstrates HtrA4 expression under hypoxic conditions and preeclampsia. (A) Downregulation of HtrA4 expression by hypoxia. BeWo cells were incubated under normoxic or hypoxic condition. After 72 hours of incubation, cells were analyzed by immunoblotting with GCM1, HtrA4, and β-actin Abs, respectively. Phase-contrast images for the morphology of normoxic and hypoxic cells were provided (left). In a separate experiment, cells were subjected to quantitative PCR analysis of the HtrA4 transcript level. (B) Suppression of HtrA4 promoter activity by hypoxia. BeWo cells were transfected with pHtrA4-1 kb and incubated under normoxic and hypoxic conditions and subsequently harvested for luciferase assays. (C) Suppression of placental cell invasion by hypoxia. BeWo cells plated into Matrigel-coated chambers were incubated under normoxic or hypoxic condition for invasion analysis. Mean values and the SD obtained from three independent experiments are presented in (A)—(C). (D) Decreased HtrA4 expression in preeclamptic placentas. Sections of two normal (N) and two preeclamptic (P) placentas at 38 weeks of gestation were immunostained with HtrA4 Ab. Note that the insets show consecutive sections immunostained with CK7 Ab. Bar, 100 μm.

FIG. 6. demonstrates regulation of syncytin-1-mediated cell-cell fusion by HtrA4. (A) HtrA4 suppresses cell-cell fusion mediated by syncytin-1. 293T cells co-expressing (a) empty vector and EGFP, (b) HtrA4-FLAG and EGFP or (c) HtrA4mt-FLAG and EGFP were co-cultured with 293T cells expressing syncytin-1-HA for 24 h. Cell-cell fusion was examined by fluorescence microscopy. (B) HtrA4 decreases the protein level of surface syncytin-1. A separate set of the cocultured 293T cells described in (A) were subjected to biotinylation, followed by streptavidin pull-down and immunoblotting with HA or EGFR mAb. As a loading control, whole cell lysates were subjected to immunoblotting with HA, FLAG, and β-actin mAbs, respectively. (C) HtrA4 regulates syncytin-1 expression in placental cells. BeWo cells stably expressing scramble or HtrA4 shRNA were subjected to biotinylation, followed by immunoprecipitation with a syncytin-1 TM Ab and then immunoblotting with HRP-conjugated streptavidin. Note that the surface EGFR protein level was not affected by HtrA4. (D) Characterization of interaction between HtrA4 and syncytin-1. Purified HtrA4mt-FLAG was incubated with agarose matrix preloaded with glutathione S-transferase (GST), GST-SU or GST-TM, followed by immunoblotting with FLAG mAb. On the other hand, HtrA4mt-FLAG or HtrA4mtδPDZ-FLAG was subjected to pull-down analysis with GST-SU demonstrating that the PDZ domain of HtrA4 is critical for recognition of the SU domain of syncytin-1.

FIG. 7. demonstrates a model of regulation of EVT differentiation by GCM1. The mononuclear cytotrophoblasts (CTs) in chorionic villi may proliferate and differentiate into a multinucleated syncytiotrophoblast (ST) layer or invasive extravillous trophoblasts (EVTs). GCM1 may regulate ST differentiation through transactivation of syncytin-1, which interacts with its cognate receptor, the sodium-dependent neutral amino acid transporter type 2 (ASCT2), to facilitate cell-cell fusion. In addition, GCM1 may participate in EVT differentiation through transactivation of HtrA4, which may cleave fibronectin in the extracellular matrix (ECM) and facilitate cell migration and invasion. HtrA4 may also cleave syncytin-1 to prevent cell-cell fusion of EVTs.

### DETAILED DESCRIPTION OF THE INVENTION

Cell-cell fusion and cell invasion are essential for placental development. Human cytotrophoblasts in the chorionic villi may undergo cell-cell fusion to form syncytiotrophoblast to facilitate nutrient-gas exchange or differentiate into extravillous trophoblasts (EVTs) to facilitate maternal-fetal circulation. The placental transcription factor, glial cell missing 1 (GCM1), regulates syncytin-1 and -2 expression to mediate trophoblast fusion. Interestingly, GCM1 and syncytin-1 are also expressed in EVTs with unknown physiological functions. A ChIP-chip analysis was performed in this study to identify HtrA4 as a GCM1 target gene, which encodes a serine protease facilitating cleavage of fibronectin and invasion of placental cells. HtrA4 was found to be immunolocalized in EVTs at the maternal-fetal interface and its expression was found to be decreased by hypoxia and in preeclampsia, a pregnancy complication associated with placental hypoxia and shallow trophoblast invasion. It was also demonstrated herein that HtrA4 interacts with syncytin-1 and suppresses cell-cell fusion. These results indicate that HtrA4 is crucial for EVT differentiation by playing a dual role in prevention of cell-cell fusion of EVTs and promotion of their invasion into the uterus.

Data from the experiments described below demonstrated that GCM 1 up-regulated the invasiveness of placental JAR and BeWo cells. A novel GCM1 target gene, HtrA4, was identified by ChIP-chip analysis. HtrA4 is a member of the high temperature requirement A (HtrA) family of serine proteases capable of cleaving the extracellular matrix (ECM) protein fibronectin and mediating JAR and BeWo cell invasion. Immunohistochemistry revealed that HtrA4 and GCM1 are coexpressed in the interstitial extravillous trophoblasts (EVTs) at the maternal-fetal interface. Moreover, HtrA4 expression was found to be decreased in BeWo cells under hypoxia and in preeclamptic placentas. The HtrA4 polypeptide is composed of an insulin growth factor binding protein domain, a Kazal protease inhibitor domain, a trypsin protease domain, and a PDZ domain (13). The studies described below reveal that HtrA4 binds to the SU subunit of syncytin-1 through its PDZ domain and that HtrA4 decreases the surface level of syncytin-1 and thereby suppresses syncytin-1-mediated cell-cell fusion. Therefore, GCM1 may regulate EVT differentiation by activating HtrA4 expression in order to stimulate EVT invasion and to counteract the fusogenic activity of syncytin-1. The studies below also reveal a novel function of GCM1 and HtrA4 in the regulation of trophoblast invasion. The results also indicate that abnormal HtrA4 expression may contribute to the development of preeclampsia. Thus, the level of HtrA4 can serve as a reliable biomarker for identifying pregnant subjects (e.g., pregnancy women) who have or are at risk for developing a pregnancy disorder. HtrA4 can also serve as a reliable marker for monitoring the development or progress of the pregnancy disorder or monitoring the efficacy of a treatment of the pregnancy disorder.

### Methods for Diagnosing Pregnancy Disorders

Some aspects of this disclosure provide diagnostic assays/methods using High Temperature Requirement A4 (HtrA4) as a biomarker to diagnose a pregnancy disorder (e.g., preeclampsia) in a pregnant subject such as a human patient. A diagnostic method/assay refers to a method or an assay for determining whether a candidate subject has, is suspected of having, or is at risk for a target disorder.

The diagnostic assays provided herein rely on the detection of the HtrA4 biomarker (either its protein level or gene transcript level). In some embodiments, the detection is qualitative, e.g., detecting the presence or absence of the HtrA4 biomarker molecule, while in other embodiments, the detection is quantitative or semi-quantitative, e.g., measuring an absolute or relative abundance of the HtrA4 biomarker molecule. In some examples, the protein level of the HtrA4 biomarker is measured, e.g., via an assay that involves an agent (e.g., antibody) that specifically recognizes HtrA4. In other examples, the level of the encoding nucleic acid (e.g., mRNA) of the HtrA4, or a fragment thereof, is measured in the methods described herein, via, e.g., an assay that involves an agent (e.g., an oligonucleotide) that specifically binds to the encoding nucleic acid (a gene transcript).

One particularly suitable type of assay for detecting, either qualitatively, semi-quantitatively, or quantitatively, is immunoassays. An immunoassay is any assay in which a target molecule (e.g., an HtrA4 biomarker molecule) is detected and/or quantified by using a binding agent as described herein that specifically binds the target molecule. The binding agent may be an antibody, which may be a full length antibody or an antigen-binding fragment thereof.

The antibody can specifically bind to HtrA4. Such an antibody may not bind to other family members such as HtrA1, HtrA2, or HtrA3.

The immunoassay may be a competitive or a non-competitive immunoassay, and may be a homogeneous or a heterogeneous immunoassay. For example, the immunoassay for detecting the HtrA4 biomarker may be an enzyme immunoassay (EIA), radioimmunoassay (RIA), fluoroimmunoassay (FIA), chemiluminescent immunoassay (CLIA), counting immunoassay (CIA), immunoenzymometric assay (IEMA), enzyme-linked immunosorbent assay (ELISA), a lateral flow immunoassay, a sandwich immunoassay, an immuno-PCR assay, a proximity ligation assay, a western blot assay, or an immunoprecipitation assay. Additional suitable immunoassays for detecting a biomarker provided herein will be apparent to those of skill in the art. It will be apparent to those of skill in the art that this disclosure is not limited to immunoassays, however, and that detection assays that are not based on an antibody or an antigen binding antibody fragment, such as mass spectrometry, are also useful for the detection and/or quantification of the HtrA4 biomarker as provided herein.

The type of detection assay used for the detection and/or quantification of the HtrA4 biomarker provided herein will depend on the particular situation in which the assay is to be used (e.g., clinical or research applications), on the kind and number of biomarkers to be detected, and on the kind and number of subject samples to be run in parallel, to name a few parameters. For example, a change in levels of HtraA4 may be detected in serum samples collected from female subjects at 10 weeks to 38 weeks of pregnancy using an immunoblotting (Western blot) assay. For example, the biological sample may be of the pregnant women at 10, 11, 12, 13, 14, 15, 16, 17, 18, 19, 20, 21, 22, 23, 24, 25, 26, 27, 29, 29, 30, 31, 32, 33, 34, 35, 36, 37 or 38 weeks of pregnancy.

While Western blot assays allow for the simultaneous analysis of the HtrA4 biomarker as well as other biomarkers of pregnancy disorders in a plurality of samples, they are limited in the number of biomarkers that may be assessed in parallel. Accordingly, in some embodiments, where a plurality of biomarkers may be analyzed in conjunction with the HtrA4 biomarker provided herein in a single sample, or in multiple samples, assays suitable for such multiplex analysis are preferred. Examples for such assays include, without limitation, peptide microarrays and lab-on-a-chip assays, which have been designed to offer a high throughput, multiplex-ready alternative to less scalable immunoassays such as Western blots (24).

To perform the diagnosis method described herein, a suitable biological sample such as a body fluid sample (e.g., blood serum, plasma, saliva, or urine), or a tissue sample, may be obtained from a candidate subject, e.g., a human pregnant woman, who may be suspected of having one or more symptoms that could be associated with a pregnancy disorder (e.g., preeclampsia or related disorders such as those described herein). The type of biological samples that is suitable for the detection of the HtrA4 biomarker will depend on the type of assay to be performed. Typically, the sample obtained from the subject will be a sample comprising the HtrA4 biomarker molecule to be assessed. In some embodiments, a suitable sample would be a blood, blood serum, or blood plasma sample comprising the HtrA4 biomarker.

The level of HtrA4 biomarker as described herein may then be determined and/or quantified via a routine method, e.g., those diagnostic assays described herein. When necessary, the data thus obtained may be normalized against the level of an internal control, which is well known in the art. The normalized level(s) of the HtrA4 biomarker may then be compared to a reference level, which may be the level of the same HtrA4 biomarker in a control sample. Optionally, the HtrA4 biomarker level in the control sample is normalized against the same internal control.

The control sample may be obtained from a female subject who is not pregnant or who is pregnant and does not have a pregnancy disorder. Alternatively, the control sample may be obtained from a pool of such subjects. Optionally, these control subjects match with the candidate subject in, e.g., age, gender, and/or ethnic background. Preferably, the control sample and the biological sample of the candidate subject are of the same type.

In some embodiments, the pregnant subject may be identified as having, suspected of having, or at risk for a pregnancy disorder as described herein (e.g., preeclampsia) if the level of HtrA4 deviates from a reference value (e.g., is lower than, or higher than, the reference value) by about 5% to about 95%, or more. In some embodiments, the pregnant subject may be identified as at risk for a pregnancy disorder (e.g., preeclampsia) if the level of HtrA4 deviates from (higher or lower than) a reference value by about 5% to about 10%, about 10% to about 20%, about 20% to about 30%, about 30% to about 40%, about 405 to about 50%, about 50% to about 60%, about 60% to about 70% about 70% to about 80%, about 80% to about 90%, or about 90% to about 100%. For example, the pregnant subject may be identified as at risk for a pregnancy disorder (e.g., preeclampsia) if the level of HtrA4 deviates from (higher or lower than) a reference value by 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In some embodiments, the levels of HtrA4 are measured over the course of pregnancy to monitor the development/progress of a pregnancy disorder such as preeclampsia. For example, at least two biological samples (e.g., serum samples or plasma samples) can be obtained from a candidate subject at different gestation stages, e.g., the first being obtained at 10-38 weeks of pregnancy and the second sample being obtained at least one weeks after obtaining the first sample (e.g., 1-30 weeks later, 1-20 weeks later, 1-10 weeks later, or 1-5 weeks later). The first biological sample may be of a pregnant women at 10, 11, 12, 13, 14, 15, 16, 17, 18, 19, 20, 21, 22, 23, 24, 25, 26, 27, 29, 29, 30, 31, 32, 33, 34, 35, 36, 37 or 38 weeks of pregnancy. In some embodiments, the second biological sample is of the pregnant subject later than 10 weeks of pregnancy. For example, the second biological sample may be of a pregnant women at 10, 11, 12, 13, 14, 15, 16, 17, 18, 19, 20, 21, 22, 23, 24, 25, 26, 27, 29, 29, 30, 31, 32, 33, 34, 35, 36, 37, 38, 39 or 40 weeks of pregnancy.

The levels of HrtA4 in the at least two biological samples can be measured as described herein. If a trend of decrease in the HrtA4 level during the course of pregnancy is observed, the candidate subject is identified as having, suspected of having, or at risk for the pregnancy disorder as described herein, such as preeclampsia. For example, the pregnant subject may be identified as having, suspected of having, or at risk for a pregnancy disorder (e.g., preeclampsia) if the level of HtrA4 in a biological sample obtained at a later gestation stage deviates from (e.g., is lower than or higher than) the level of HtrA4 in a biological sample obtained at an earlier gestation stage (e.g., deviates by about 5% to about 95%, about 5% to about 10%, about 10% to about 20%, about 20% to about 30%, about 30% to about 40%, about 405 to about 50%, about 50% to about 60%, about 60% to about 70% about 70% to about 80%, about 80% to about 90%, or about 90% to about 100%). For example, the pregnant subject may be identified as having, suspected of having, or at risk for a pregnancy disorder (e.g., preeclampsia) if the level of HtrA4 at a later gestation stage deviates from (higher or lower than) a level of HtrA4 at an earlier gestation stage by 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

If the pregnant subject has been diagnosed as having the pregnancy disorder, a decrease trend of HtrA4 during the course of the pregnancy (e.g., the level of HtrA4 in the second sample is lower than that in the first sample by at least 20%, e.g., 30%, 40%, 50%, 60%, 70%, 80%, 90%, 1-fold, 2-fold, 5-fold, 10-fold, 50-fold, or 100-fold) indicates disease progresses.

When necessary, any of the diagnostic methods/assays described herein can involve HrtA4 in combination with one or more additional biomarkers. If the level of HrtA4 deviates from a reference value (e.g., is lower than/or decreases or is higher than/increases) over the course of pregnancy and the level of the one or more additional biomarker(s) differ(s) from a reference level (e.g., lower or higher) or changes over the course of pregnancy, the candidate subject is diagnosed as having, suspected of having, or at risk for a pregnancy disorder as described herein.

When more than one pregnancy disorder biomarker is investigated, the levels of the biomarkers (preferably normalized against an internal control) may be processed by, e.g., a computational program to generate an expression profile (e.g., a biomarker signature), which may be represented by a number or numbers, that characterize a pattern of the biomarkers in the biological sample. The levels of the same biomarkers from the control sample may be processed by the same method to generate a reference signature. The biomarker signature of the candidate subject may be compared with the control signature to determine whether the candidate subject has, is suspected of having, or is at risk for a pregnancy disorder (e.g., preeclampsia).

Various computational programs may be applied in the methods of this disclosure to aid in analysis of expression data. Examples include, but are not limited to, Prediction Analysis of Microarray (PAM; see Tibshirani et al., PNAS 99(10):6567-6572, 2002); Plausible Neural Network (PNN; see, e.g., U.S. Pat. No. 7,287,014), PNNSulotion software and others provided by PNN Technologies Inc., Woodbridge, Va., USA, and Significance Analysis of Microarray (SAM).

The differences between the biomarker level/signature of a candidate subject and the control sample preferably are statistically significant. Statistical significance may be determined by any art known method. Exemplary statistical tests include: the Students T-test, Mann Whitney U non-parametric test, and Wilcoxon non-parametric statistical test. Some statistically significant relationships have a P value of less than 0.05 or 0.02. Particular binding proteins may show a difference, e.g., in specificity or binding that is statistically significant (e.g., P value<0.05 or 0.02). The terms “induce”, “inhibit”, “potentiate”, “elevate”, “increase”, “decrease” or the like, e.g., which denote distinguishable qualitative or quantitative differences between two states, and may refer to a difference, e.g., a statistically significant difference, between the two states.

Examples of pregnancy disorders that may be identified in accordance with the diagnostic methods/assays described herein include, without limitation, an endocrine disorder (e.g., diabetes mellitus, type I, gestational diabetes, thyroid disease, hyperparathyroidism, pituitary adenoma), a renal disorder, a hypertensive disorder (e.g., preeclampsia, eclampsia, chronic hypertension, late or transient hypertension), a cardiovascular disorder, a thromboembolic disorder, a hepatic disorder, a rheumatic disorder, a hematologic disorder, a neurologic disorder, a digestive tract disorder, or a combination of the foregoing disorders. In some embodiments, the pregnancy disorder is preeclampsia.

After a pregnant subject such as a human patient has been identified as having, suspected of having, or at risk for any of the pregnancy disorders described herein (e.g., preeclampsia), a such a subject can be subjected to a further test (e.g., blood pressure measurement or blood clotting test) to confirm disease occurrence and/or suitable therapy. Alternatively, procedures such as monitoring symptoms associated with the pregnancy disorder and/or monitoring the health of the baby can be performed to the subject show has been diagnosed by the methods/assays described herein as having, suspected of having, or at risk for the pregnancy disorder. For example, if a candidate subject is diagnosed as having, suspected of having, or at risk for preeclampsia, one or more physical examinations can be performed on the subject after the diagnosis, including blood pressure measurement, examining swelling in the hands and fact, monitoring weight gain, examining protein in the urine, investigating liver enzyme levels, measuring platelet count, testing blood clot, and or monitoring the baby's health. When necessary, the subject can be subjected to bed resting and diet control to reduce salt intake, or medications such as taking blood pressure-control drug or steroid to speed up development of the baby's lung.

In some embodiments, the methods described herein can further comprise treating the pregnant subject to alleviate a symptom associated with the pregnancy disorder, if the subject is diagnosed as having or at risk for the disorder. Such treatments include, but are not limited to, smoking cessation, antihypertensive therapy, magnesium sulfate treatment, dietary and nutritional factor treatment, aspirin supplementation treatment, exercise, induction of paternal tolerance, or administration of immune factors.

In other embodiments, the methods described herein further comprises delivering the baby, if the subject is diagnosed as having a pregnancy disorder (e.g., preeclampsia) at a severe level.

### Diagnostic Binding Agents

The diagnostic methods described herein may involve one or more binding agents each specifically binding the HtrA4 biomarker used in the methods provided herein. An agent “specifically binds” to a target biomarker if it binds with greater affinity, avidity, more readily, and/or with greater duration than it binds to other substances. For example, an antibody (e.g., an anti-HtrA4 antibody) that specifically (or preferentially) binds to an antigen epitope (e.g., an epitope of HtrA4) is an antibody that binds this epitope with greater affinity, avidity, more readily, and/or with greater duration than it binds to other antigen epitopes (e.g., non-HtrA4 epitopes). It is also understood by reading this definition that, for example, an antibody that specifically binds to a first target antigen may or may not specifically or preferentially bind to a second target antigen. As such, “specific binding” or “preferential binding” does not necessarily require (although it may include) exclusive binding. Generally, but not necessarily, reference to binding means preferential binding.

Suitable binding agents typically bind their target molecule, for example the HtrA4 biomarker molecule, with high affinity and/or high specificity. Typically, high affinity binding agents provided herein are binding agents that bind their respective target molecule with a dissociation constant (Kd) of <10⁻⁷, <10⁻⁸, <10⁻⁹, <10⁻¹⁰, or <10⁻¹¹. High-specificity binding agents provided herein bind their target molecule with high affinity but do not bind to any molecule other than their target molecule, or bind a molecule other than their target molecule with low affinity. For example, some high-specificity binding agents provided herein bind their target molecule (e.g., HtrA4 biomarker) with high affinity, e.g., with a dissociation constant (Kd) of <10⁻⁷, <10⁻⁸, <10⁻⁹, <10⁻¹⁰, or <10⁻¹¹, but do not bind any molecule other than their target molecule, or bind a molecule other than their target molecule with a much lower affinity, e.g., with a dissociation constant (Kd) of >10⁻⁷, >10⁻⁶, >10⁻⁵, >10⁻⁴, >10⁻³, or >10⁻².

Binding affinity may be determined herein by a variety of methods including equilibrium dialysis, equilibrium binding, gel filtration, ELISA, surface plasmon resonance, or spectroscopy (e.g., using a fluorescence assay). Exemplary conditions for evaluating binding affinity are in PBS (phosphate buffered saline) at pH 7.2 at 30° C. These techniques may be used to measure the concentration of bound and free binding protein as a function of binding protein (or target) concentration. The concentration of bound binding protein ([Bound]) is related to the concentration of free binding protein ([Free]) and the concentration of binding sites for the binding protein on the target where (N) is the number of binding sites per target molecule by the following equation:

[Bound]=N·[Free]/((1/Ka)+[Free]).

It is not always necessary to make an exact determination of Ka. In some instances, it is sufficient to obtain a quantitative measurement of affinity (e.g., determined using a method such as ELISA or FACS analysis) that is proportional to Ka, and thus may be used for comparisons, such as determining whether a higher affinity is, e.g., 2 fold higher, to obtain a qualitative measurement of affinity, or to obtain an inference of affinity (e.g., by activity in a functional assay, e.g., an in vitro or in vivo assay).

In some embodiments, the binding agent is an antibody that specifically binds the HtrA4 biomarker. Alternatively, the binding agent may be a non-antibody binding agent, for example, a DARPin (designed ankyrin repeat proteins), an AdNectin, an Affibody or other antibody mimetic, an Anticalin, an Avimer, an Aptamer, a Ligand, or an inert enzyme substrate. Additional suitable binding agents will be apparent to those of skill in the art, and the invention is not limited in this respect.

Diagnostic antibodies useful in the methods described herein may be antibodies that specifically bind to the HtrA4 biomarker and may be used for, e.g., for detection and/or quantification.

An antibody (interchangeably used in plural form) is an immunoglobulin molecule capable of specific binding to a target such as, for example, a carbohydrate, polynucleotide, lipid, or polypeptide through at least one antigen recognition site, located in the variable region of the immunoglobulin molecule. As used herein, the term “antibody” encompasses not only intact (i.e., full-length) polyclonal or monoclonal antibodies, but also antigen-binding fragments thereof (such as, for example, Fab, Fab′, F(ab′)₂, Fv), single chain (scFv), mutants thereof, fusion proteins comprising an antibody portion, humanized antibodies, chimeric antibodies, diabodies, linear antibodies, single chain antibodies, multispecific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies.

An antibody includes an antibody of any class such as, for example, IgD, IgE, IgG, IgA, or IgM (or sub-class thereof), and the antibody need not be of any particular class. Depending on the antibody amino acid sequence of the constant domain of its heavy chains, immunoglobulins may be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

The antibodies to be used in the methods described herein may be murine, rat, human, or any other origin (including chimeric or humanized antibodies). In some examples, the antibody comprises a modified constant region, such as a constant region that is immunologically inert, e.g., does not trigger complement mediated lysis, or does not stimulate antibody-dependent cell mediated cytotoxicity (ADCC). ADCC activity may be assessed using methods disclosed in U.S. Pat. No. 5,500,362. In other embodiments, the constant region is modified as described in Eur. J. Immunol. (1999) 29:2613-2624; PCT Application No. PCT/GB99/01441; and/or UK Patent Application No. 9809951.8.

Furthermore, although the two domains of the Fv fragment, V_{L} and V_{H}, are coded for by separate genes, they may be joined, using recombinant methods, by a synthetic linker that enables them to be made as a single protein chain in which the VL and VH regions pair to form monovalent molecules known as single chain Fv (scFv). See e.g., Bird et al. (1988) Science 242:423-426; and Huston et al. (1988) Proc. Natl. Acad. Sci. USA 85:5879-5883.

Antibodies suitable for use in the diagnostic methods described herein may be made by any method known in the art. See, for example, Harlow and Lane, (1988) Antibodies: A Laboratory Manual, Cold Spring Harbor Laboratory, New York. Antibodies that specifically bind the HtrA4 biomarker described herein are also commercially available.

In some embodiments, antibodies specific to the HtrA4 biomarker may be made by the conventional hybridoma technology. The full-length target antigen or a fragment thereof, optionally coupled to a carrier protein such as HtrA4, may be used to immunize a host animal for generating antibodies binding to that antigen. The route and schedule of immunization of the host animal are generally in keeping with established and conventional techniques for antibody stimulation and production, as further described herein. General techniques for production of mouse, humanized, and human antibodies are known in the art and are described herein. It is contemplated that any mammalian subject including humans or antibody producing cells therefrom may be manipulated to serve as the basis for production of mammalian, including human hybridoma cell lines. Typically, the host animal is inoculated intraperitoneally, intramuscularly, orally, subcutaneously, intraplantar, and/or intradermally with an amount of immunogen, including as described herein.

Hybridomas may be prepared from the lymphocytes and immortalized myeloma cells using the general somatic cell hybridization technique of Kohler, B. and Milstein, C. (1975) Nature 256:495-497 or as modified by Buck, D. W., et al., In Vitro, 18:377-381 (1982). Available myeloma lines, including but not limited to X63-Ag8.653 and those from the Salk Institute, Cell Distribution Center, San Diego, Calif., USA, may be used in the hybridization. Generally, the technique involves fusing myeloma cells and lymphoid cells using a fusogen such as polyethylene glycol, or by electrical means well known to those skilled in the art. After the fusion, the cells are separated from the fusion medium and grown in a selective growth medium, such as hypoxanthine-aminopterin-thymidine (HAT) medium, to eliminate unhybridized parent cells. Any of the media described herein, supplemented with or without serum, may be used for culturing hybridomas that secrete monoclonal antibodies. As another alternative to the cell fusion technique, EBV immortalized B cells may be used to produce the anti-HtrA4 biomarker monoclonal antibodies of the subject invention. The hybridomas are expanded and subcloned, if desired, and supernatants are assayed for anti-immunogen activity by conventional immunoassay procedures (e.g., radioimmunoassay, enzyme immunoassay, or fluorescence immunoassay).

Hybridomas that may be used as source of antibodies encompass all derivatives, progeny cells of the parent hybridomas that produce monoclonal antibodies capable of binding to the HtrA4 biomarker. Hybridomas that produce such antibodies may be grown in vitro or in vivo using known procedures. The monoclonal antibodies may be isolated from the culture media or body fluids, by conventional immunoglobulin purification procedures such as ammonium sulfate precipitation, gel electrophoresis, dialysis, chromatography, and ultrafiltration, if desired. Undesired activity if present, may be removed, for example, by running the preparation over adsorbents made of the immunogen attached to a solid phase and eluting or releasing the desired antibodies off the immunogen. Immunization of a host animal with a target antigen or a fragment containing the target amino acid sequence conjugated to a protein that is immunogenic in the species to be immunized, e.g., keyhole limpet hemocyanin, serum albumin, bovine thyroglobulin, or soybean trypsin inhibitor using a bifunctional or derivatizing agent, for example maleimidobenzoyl sulfosuccinimide ester (conjugation through cysteine residues), N-hydroxysuccinimide (through lysine residues), glutaraldehyde, succinic anhydride, SOCl, or R1N═C=NR, where R and R1 are different alkyl groups, may yield a population of antibodies (e.g., monoclonal antibodies).

If desired, an antibody (monoclonal or polyclonal) of interest (e.g., produced by a hybridoma) may be sequenced and the polynucleotide sequence may then be cloned into a vector for expression or propagation. The sequence encoding the antibody of interest may be maintained in vector in a host cell and the host cell may then be expanded and frozen for future use. In an alternative, the polynucleotide sequence may be used for genetic manipulation to “humanize” the antibody or to improve the affinity (affinity maturation), or other characteristics of the antibody. For example, the constant region may be engineered to more resemble human constant regions to avoid immune response if the antibody is used in clinical trials and treatments in humans. It may be desirable to genetically manipulate the antibody sequence to obtain greater affinity to the target antigen and greater efficacy. It will be apparent to one of skill in the art that one or more polynucleotide changes may be made to the antibody and still maintain its binding specificity to the target antigen.

Antigen-binding fragments of an intact antibody (full-length antibody) may be prepared via routine methods. For example, F(ab′)2 fragments may be produced by pepsin digestion of an antibody molecule, and Fab fragments that may be generated by reducing the disulfide bridges of F(ab′)2 fragments.

Alternatively, an antibody capable of binding to the HtrA4 biomarker may be identified by screening a suitable antibody library, such as a phage-display or yeast-display library. See, e.g., U.S. Pat. Nos. 4,946,778 and 4,704,692.

### Diagnostic Devices

Some aspects of this disclosure provide diagnostic devices (e.g., diagnostic chips) for the detection and/or quantification of the HtrA4 biomarker provided herein. In some embodiments, a diagnostic device is provided that comprises a solid support, to which one or more binding agents that specifically binds an HtrA4 biomarker is conjugated. In some embodiments, the solid support is or is part of a plate that may be used in immunoassay, for example, an optical plate or an ELISA plate. In some such embodiments, the binding agent is conjugated to the bottom of a plate well. In some embodiments, the solid support is part of a diagnostic chip, for example, a microarray chip, a microfluidics chip, or a lab-on-a-chip chip.

In some embodiments, the diagnostic device provided herein is in the form of a diagnostic chip. A diagnostic chip may, in some embodiments, comprise a surface, such as a glass or plastic surface, to which a plurality of binding agents each specifically binding an HtrA4 biomarker and, optionally, one or more additional biomarker(s) of a pregnancy disorder (e.g., preeclampsia) is conjugated. Typically, the different binding agents will be conjugated to the chip surface in a spatially distinct manner, so that each binding agent occupies a specific, identifiable area, for example, a distinct spot, on the chip surface. The chip surface comprising the conjugated binding agents may then be contacted with a sample obtained from the subject under conditions suitable for any biomarker molecules that may be present in the sample tube binds to the respective binding agent on the surface of the chip. After washing away of any unbound molecules, any bound HtrA4 biomarker molecules (and any other biomarker(s) of a pregnancy disorder) may then be detected using a suitable detection agent and method. Suitable detection agents a and methods for the detection of bound biomarker molecules are well known to those of skill in the art, include, but are not limited to, for example, sandwich detection assays, secondary antibody assays, and enzyme-linked detection assays. The invention is not limited in this respect.

A diagnostic device provided herein that allows for a quantitative or semiquantitative measurement of the HtrA4 biomarker may also comprise a binding agent useful for standardization of the measured value(s) of the HtrA4 biomarker. A diagnostic device provided herein may further comprise a binding agent that is useful to generate a reference or control value.

Without further elaboration, it is believed that one skilled in the art may, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Materials and Methods

### Plasmid Constructs

A DNA fragment encoding wild-type HtrA4 fused with a C-terminal FLAG tag was cloned into pcDNA3 (Invitrogen, Carlsbad, Calif.) to generate the pHtrA4-FLAG expression plasmid.

A serine-to-alanine mutation was introduced into the Ser326 residue, which is the active site of HtrA4, to generate the pHtrA4mt-FLAG expression plasmid for producing a protease-dead HtrA4 mutant.

The pHtrA4mtδPDZ-FLAG expression plasmid was generated by deletion of the PDZ domain in the HtrA4mt cDNA. Human GCM1 cDNA fragment fused with an N-terminal hemagglutinin (HA) tag was subcloned into pcDNA3 to generate the pHA-GCM1 expression plasmid.

For lentiviral expression, cDNA fragments for HA-GCM1, HtrA4-FLAG, and HtrA4mt-FLAG were subcloned into pCDH (SBI, Mountain View, Calif.) to generate pCDH-HA-GCM1, pCDH-HtrA4-FLAG, and pCDH-HtrA4mt-FLAG, respectively. For RNA interference, lentiviral pLKO.1-Puro shRNA expression plasmids harboring a scramble sequence (5′-CCTAAGGTTAAGTCGCCCTCG-3′) (SEQ ID NO:1) and sequences for GCM1 (5′-CCTCAGCAGAACTCACTAAAT-3′) (SEQ ID NO:2) and HtrA4 (5′-AAGCTACATACCC AGCCCTCC-3′) (SEQ ID NO:3), respectively, were provided by the National RNAi Core Facility of Taiwan.

Human genomic DNA fragment containing the HtrA4 promoter region from nucleotide (nt)-971 to 29 (relative to the translational initiation site) was subcloned into pGL3E1B to generate the reporter construct, pHtrA4-1 kb. A proximal GCM 1-binding site (GBS, nt-284 to -277) in the abovementioned HtrA4 promoter region was mutated to generate the pHtrA4-1 kb-GBSmt reporter construct. The pSyn1-HA expression plasmid, which encodes syncytin-1 with a C-terminal HA tag (syncytin-1-HA), has been described previously (11).

### Cell Culture, Transfection, and Lentivirus Transduction

The 293T, JAR, and BeWo cells were obtained from the American Type Culture Collection (ATCC) (Manassas, Va.). For transient expression, cells were transfected with the indicated reporter and expression plasmids using Lipofectamine 2000 reagent (Invitrogen). For stable expression of exogenous HA-GCM1, HtrA4-FLAG, and HtrA4mt-FLAG, cells were infected with the recombinant lentivirus strains harboring the aforementioned pCDH constructs. On the other hand, stable GCM1- and HtrA4-knockdown cells were established using the recombinant lentivirus strains harboring the aforementioned pLKO.1-Puro shRNA expression plasmids. The infected cells were subjected to antibiotic selection using 1 μg/ml of puromycin and the puromycin-resistant clones were pooled for studies. To study the effects of hypoxia on HtrA4 expression and promoter activity and cell invasion, cells were incubated under hypoxic (1% O₂, 5% CO₂, and 94% N₂) or normoxic (21% O₂, 5% CO₂, and balance N₂) conditions at 37° C. for 72 hours prior to further analysis.

### Chromatin Immunoprecipitation-on-Chip (ChIP-Chip) Analysis

BeWo cells stably expressing HA-GCM1 (BeWo31) (36) were treated with or without 50 μM forskolin for 24 hours before being subjected to ChIP assay using normal mouse serum or HA mAb (Sigma, St. Louis, Mo.). The immunopurified genomic fragments were amplified by ligation-mediated PCR and the resulting amplicons were fragmented and labeled with terminal labeling kit (Affymetrix, Santa Clara, Calif.) according to the manufacturer's instructions. The chips of Human promoter 1.0R array (Affymetrix) were hybridized with the labeled amplicons, followed by data analysis using the Partek software package (St. Louis, Mo.). Furthermore, RefSeq mapping data were collected from the UCSC Genome Browser's RefFlat table (The UCSC genome browser database: update 2007). The closest RefSeq 5′ end for each ChIP region was identified, regardless of whether the cluster was upstream or downstream of the 5′ end. The primer sequences for ChIP analysis of the interaction between GCM1 and HtrA4-GBS in BeWo cells are 5′-TGGAAACTGTTACGCTTCTCA-3′ (SEQ ID NO:4) and 5′-GTCTCTAGCC CTACCCG-3′ (SEQ ID NO:5). On the other hand, analysis of direct interaction between GCM1 and HtrA4-GBS was performed by electrophoretic mobility shift assay (EMSA) as previously described (22). In brief, recombinant GCM1-FLAG (8) was incubated with a radiolabeled oligonucleotide probe harboring the wild-type (5′-GTGAGCTTTCTTATCAGTC TGCCCTCATTGTCGGTTTT-3′) (SEQ ID NO:6) or mutant (5′-GTGAGCTTTCTTATCAG TCTGAACTACTTGTCGGTTTT-3′) (SEQ ID NO:7) HtrA4-GBS in the presence of GCM1 or unrelated syncytin-2 Ab (11). The reaction mixtures were analyzed by electrophoresis on 5% non-denaturing polyacrylamide gels.

### Cell Invasion Assay

Cell invasion analysis of GCM1- or HtrA4-expressing JAR cells and GCM 1- or HtrA4-knockdown BeWo cells were performed using Matrigel invasion chambers (BD Biosciences, Bedford, Mass.) according to the manufacturer's instructions. Briefly, cells were plated into the chambers and incubated for 48 h. Migrated cells in the lower surface of the filters were fixed with paraformaldehyde and visualized by crystal violet stain and counted. Four microscopic fields per sample were randomly selected for quantification in each of three independent experiments. Images were prepared for presentation using Adobe Photoshop v7.0. Immunohistochemistry, flow cytometry analysis, and single cell RT-PCR.

Immunohistochemicstry, flow cytometry analysis and single cell RT-PCT

Normal and preeclamptic placental tissue biopsies were fixed in neutral buffered formalin, embedded in paraffin, and sectioned. Tissue sections were de-paraffinized and subjected to immunostaining by incubation with normal serum, cytokeratin 7 (CK7, Millipore, Billerica, Mass.), HtrA4, and GCM1 Abs, respectively. The sections were then incubated sequentially with biotinylated secondary antibody and horseradish peroxidase (HRP)-conjugated streptavidin. Antigenic detection was performed using chromogenic substrate 3,3′-diaminobenzidine tetrahydrochloride (DAB) and the sections were further counterstained by hematoxylin. The HtrA4 antibody was prepared from guinea pigs immunized with His-tagged recombinant protein containing the region of amino acids 170-476 of HtrA4 polypeptide.

To purify EVTs, villous tissues in the basal plate of term placenta were collected, trypsinized, and subjected to Percoll gradient centrifugation to enrich trophoblast cells (11). The trophoblast cells were then incubated with HLA-G Ab (Abeam, Cambridge, Mass.) and Alexa Fluor® 488-conjugated secondary Ab (Invitrogen) and subsequently analyzed in a FACSAria II flow cytometer (BD Biosciences) to obtain the HLA-G-positive EVTs. Increasing number of EVTs (5, 50, 500 cells) were lysed using a RealTime ready cell lysis kit (Roche Applied Science, Indianapolis, Ind.) and reversely transcribed, followed by analysis of GCM 1 and HtrA4 transcripts in a LightCycler 480 real-time PCR instrument (Roche Applied Science). Sequence of the primer sets for PCR analysis in this study are HtrA1 5′-GGACTACATCCAGACCGAC-3′ (SEQ ID NO:8) and 5′-TGGGACTCCGTGAGGAAC-3′ (SEQ ID NO:9) for HtrA1, 5′-TCTGGAGGTCCCCTGGTTA-3′ (SEQ ID NO:10) and 5′-AGCATCATCACCCCAATGT-3′ (SEQ ID NO:11) for HtrA2,5′-GAGGGCTGGTCACATGAAGA-3′ (SEQ ID NO:12) and 5′-GCTCCGCTAATTTCCAGT-3′ (SEQ ID NO:13) for HtrA3,5′-GTCAGCACCAAACAGCG-3′ (SEQ ID NO:14) and 5′-GGAGATTCCATCAGTCACCC-3′ (SEQ ID NO:15) for HtrA4,5′-CTGACAAGGCTTTTTTCTTCACA-3′ (SEQ ID NO:16) and 5′-CCAGACGGGACAGGTTT-3′ (SEQ ID NO:17) for GCM1, and 5′-AACTCCATCATGAAGTGTGACG-3′ (SEQ ID NO:18) and 5′-GATCCACATCTGCTGGAAGG-3′ (SEQ ID NO:19) for β-actin.

### Preparation of GST Fusion Protein and HtrA4 Recombinant Protein

The GST fusion proteins GST-SU and GST-TM, which contain the SU and the TM subunits of syncytin-1, respectively, were prepared in E. coli using the pGEX6P-1 vector (GE Healthcare, Piscataway, N.J.). Recombinant HtrA4-FLAG and HtrA4mt-FLAG proteins were prepared in a vaccinia virus expression system. In brief, 293T cells were infected with vTF7-3, a vaccinia virus expressing T7 RNA polymerase, followed by transfection of pHtrA4-FLAG, pHtrA4mt-FLAG, and pHtrA4mt6PDZ=FLAG, respectively. Recombinant HtrA4-FLAG, HtrA4mt-FLAG, and HtrA4mtδPDZ-FLAG proteins were purified from the culture media of transfected 293T cells using agarose beads conjugated with FLAG mAb (Sigma). Cleavage of ECM protein by HtrA4 was performed by incubation of fibronectin (Calbiochem, La Jolla, Calif.) and HtrA4-FLAG or HtrA4mt-FLAG at 37° C. for 16 h, followed by immunoblotting with fibronectin Ab (Sigma). Pull-down experiments were performed by incubation of GST-SU- or GST-TM-preloaded glutathione agarose beads with HtrA4mt-FLAG to identify the interaction domain in syncytin-1 for HtrA4. Likewise, GST-SU-preloaded glutathione agarose beads were incubated with HtrA4mt-FLAG or HtrA4mt□PDZ-FLAG to identify the interaction domain in HtrA4 for syncytin-1. HtrA4mt-FLAG, instead of wild-type HtrA4-FLAG, was used in the pull-down experiments to prevent cleavage of the GST fusion proteins.

### Cell-Cell Fusion Analysis and Analysis of Surface Syncytin-1

To test the effect of HtrA4 on syncytin-1-mediated cell-cell fusion, 293T cells were first infected with the vTF7-3 vaccinia virus. The infected 293T cells were then split into different populations for transfection of the green fluorescent protein expression plasmid pEGFP—N 1 (Clontech, Mountain View, Calif.) plus (i) pcDNA3, (ii) pHtrA4-FLAG, or (iii) pHtrA4mt-FLAG.

An additional population of infected 293T cells was transfected with pSyn1-HA. The 293T cells expressing syncytin-1-HA were co-cultured with the 293T cells co-expressing EGFP plus the wild-type or mutant HtrA4. After 16 h, cell-cell fusion was examined under an Olympus immunofluorescence microscope (Tokyo, Japan) equipped with a cooled charge-coupled device camera (DP50). To analyze the syncytin-1-HA on cell surfaces, a separate set of the aforementioned co-cultured cells was subjected to surface biotinylation using sulfosuccinimidyl 2-(biotinamido)-ethyl-1,3′-dithiopropionate (sulfo-NHS—SS-biotin, Thermo Scientific, Rockford, Ill.). Streptavidin-conjugated agarose beads were added to pull down the biotinylated proteins, followed by immunoblotting with HA mAb. Likewise, BeWo cells expressing scramble or HtrA4 shRNA were subjected to biotinylation reaction, followed by immunoprecipitation with an antibody against the TM subunit of syncytin-1 (7) and immunoblotting with HRP-conjugated streptavidin.

### Results

### GCM1 Regulates Placental Cell Invasion

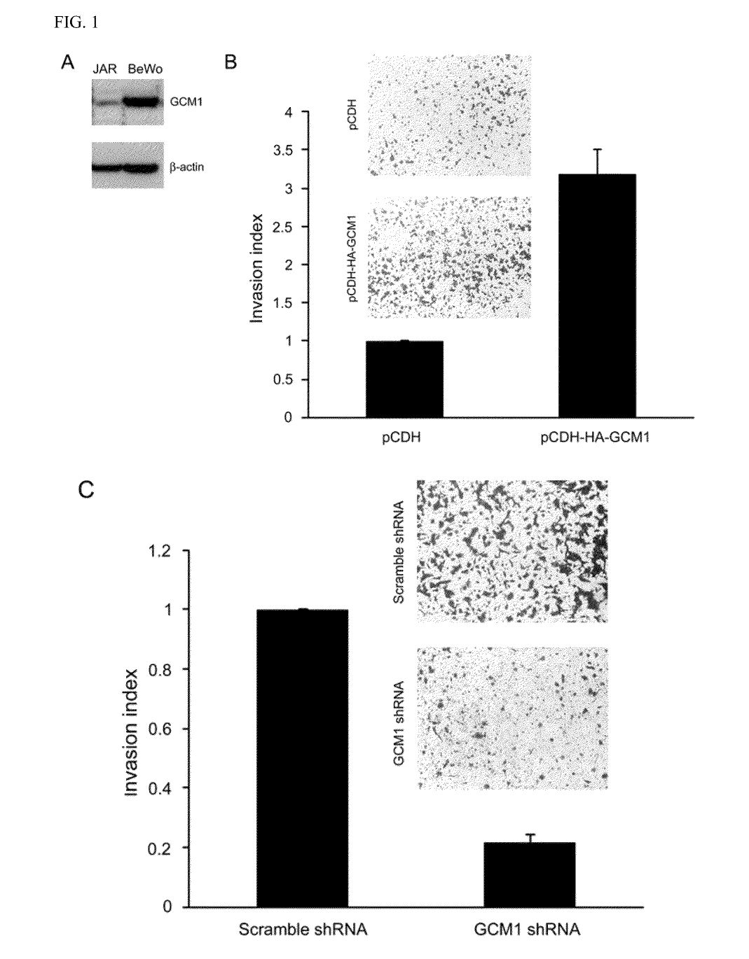

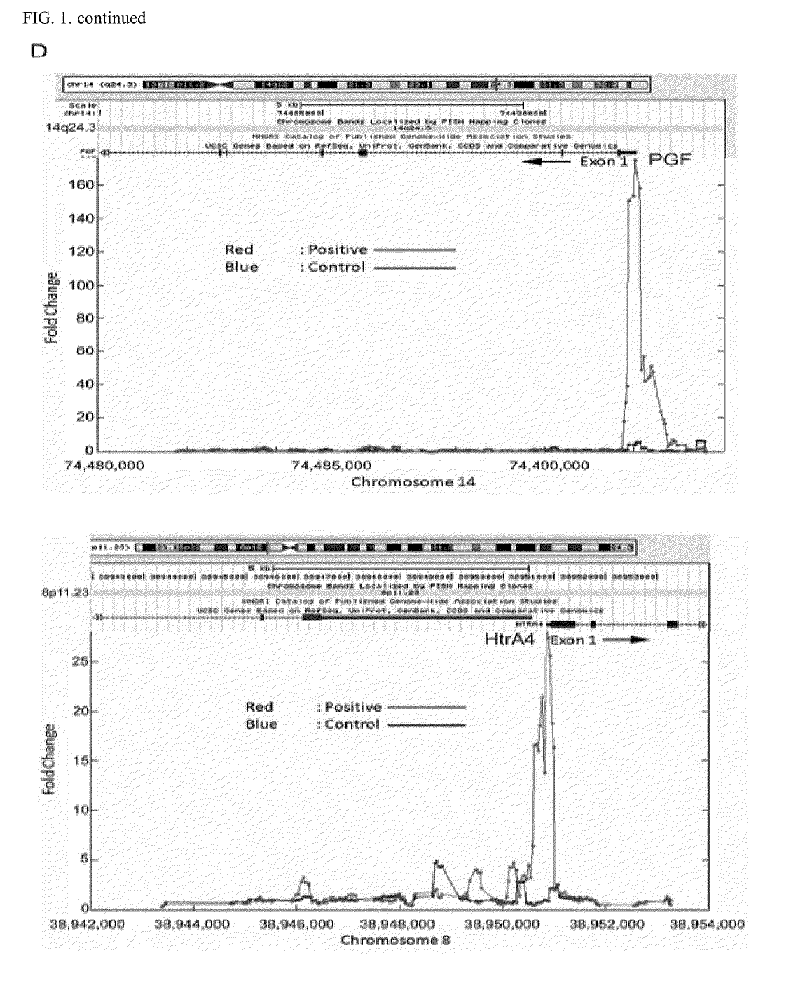

The discovery that GCM1 is expressed in EVTs raises the possibility that GCM1 may play an important role in regulating trophoblast invasion. To test this hypothesis, whether GCM1 regulates the invasion activity of placental JAR and BeWo cells was examined. As the endogenous GCM1 protein level is much lower in JAR cells relative to that in BeWo cells (FIG. 1A), exogenous HA-tagged GCM1 (HA-GCM1) was introduced into JAR cells using a lentiviral expression system to establish JAR cells stably expressing HA-GCM 1. For invasion analysis, mock (harboring the pCDH empty vector) and HA-GCM1-expressing JAR cells were plated into Matrigel-coated transwells. As shown in FIG. 1B, invasion of the HA-GCM1-expressing JAR cells was significantly enhanced in comparison with the mock cells. As a complementary approach, the effect of GCM1 knockdown on the invasion activity of BeWo cells was investigated. The results indicate that, as compared with BeWo cells stably expressing scramble short-hairpin (shRNA), BeWo cells stably expressing GCM1 shRNA exhibited much lower invasion activity (FIG. 1C). These results indicate that GCM 1 may participate in placental cell invasion.

### HtrA4 is a GCM1 Target Gene

GCM1 may transactivate novel target genes encoding the critical downstream effectors in the regulation of placental cell invasion. ChIP-chip experiments were performed using BeWo31 cells, which stably express HA-GCM1, to identify the candidate GCM1 target genes involved in placental cell invasion. As expected, placental growth factor (PGF) was identified as one of the GCM1 target genes in the ChIP-chip experiments (FIG. 1D, left). Unexpectedly, HtrA4 was identified as a novel GCM1 target gene (FIG. 1D, right). HtrA4 encodes a serine protease primarily expressed in placenta. The role of this protein in regulating placental cell invasion was further investigated.

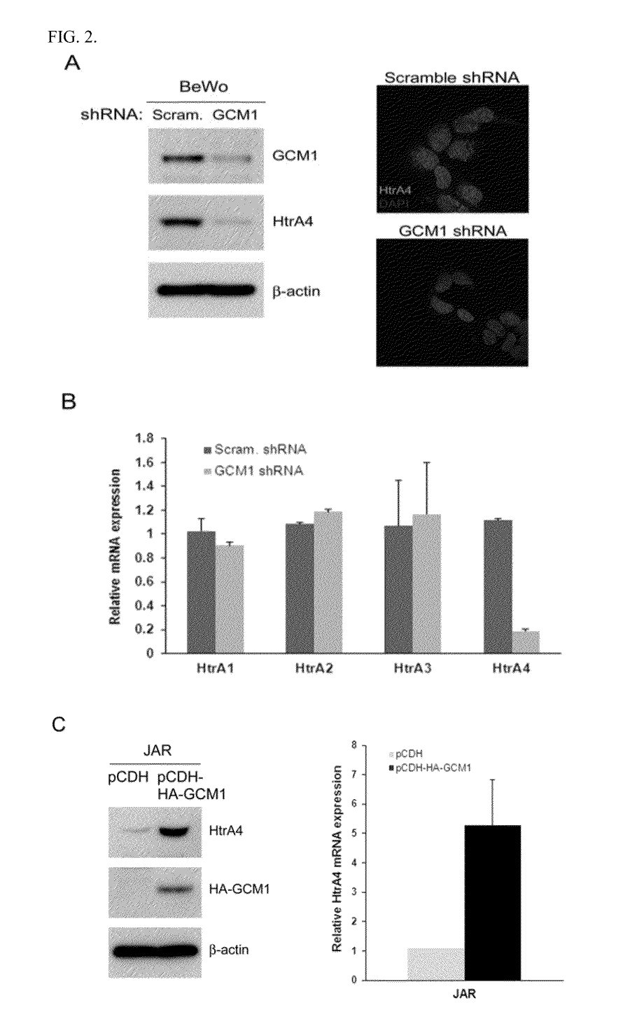

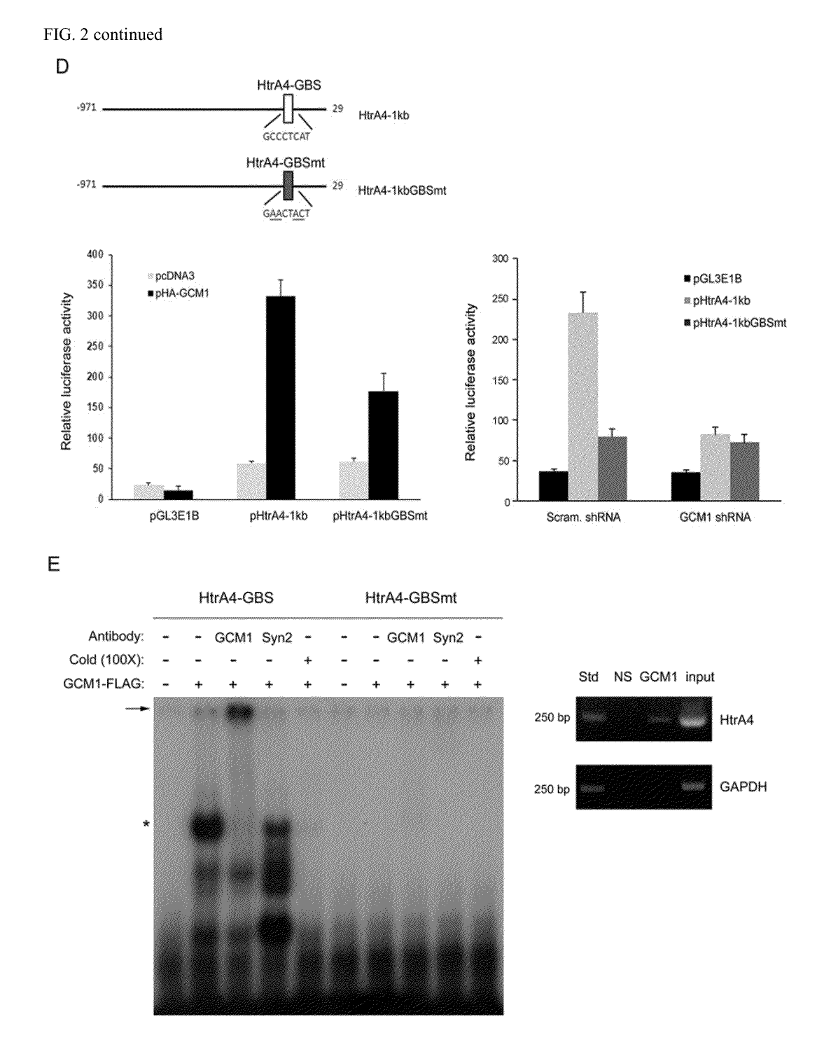

The regulatory activity of GCM1 on HtrA4 expression was examined to confirm that HtrA4 is a bona fide target gene of GCM1. An HtrA4 antibody was generated that does not cross-react with the other HtrA family members. The HtrA4 protein levels in the GCM1 knockdown and scramble control BeWo cells were measured using this Htr4A-specific antibody. As shown in the left panel of FIG. 2A, the HtrA4 protein level measured by the immunoblotting assay described herein was significantly decreased by knocking down GCM1. Consistently, lower HtrA4 levels were detected in the cytoplasm of GCM1-knockdown BeWo cells by immunofluorescence microscopy (FIG. 2A, right). Moreover, the transcript level of HtrA4, but not HtrA 1, -2, and -3, was also significantly decreased by GCM1 knockdown (FIG. 2B). Immunoblotting and quantitative polymerase chain reaction (PCR) analyses were also performed in the HA-GCM1-expressing and mock JAR cells for measuring HtrA4 expression. The results show that GCM1 overexpression positively stimulated HtrA4 expression at both the protein and transcript levels in JAR cells (FIG. 2C).

Whether GCM1 directly regulates the promoter activity of HtrA4 was investigated by transient expression experiments. The pHtrA4-1 kb reporter plasmid, which harbors a 1 kb HtrA4 promoter region proximal to the translational initiation site, was co-transfected with pHA-GCM1 into 293T cells. The luciferase reporter activity directed by pHtrA4-1 kb was positively stimulated by HA-GCM1 (FIG. 2D, left panel). In line with the results from the ChIP-chip assay described above, a potential GCM1-binding site (HtrA4-GBS) was identified within the 1 kb HtrA4 promoter region (FIG. 2D, upper panel). When HtrA4-GBS was mutated in the pHtrA4-1kbGBSmt, the luciferase reporter activity stimulated by GCM1 was compromised (FIG. 2D, left panel), indicating that the identified HtrA4-GBS is a functional GCM1-responsive element in the HtrA4 promoter. This was further supported by the fact that the luciferase activity directed by pHtrA4-1 kb, but not pHtrA4-1 kbGBSmt, was significantly decreased in the GCM1-knockdown BeWo cells (FIG. 2D, right panel). Of note, the possibility of additional binding sites for GCM1 and/or GCM 1-interacting factors within the proximal 1 kb region of HtrA4 promoter cannot be ruled out as GCM1 still partially stimulates the reporter activity directed by pHtrA4-1 kbGBSmt (FIG. 2D, left panel).

Next, EMSA was performed to characterize the interaction between GCM 1 and HtrA4-GBS. To this end, recombinant GCM1-FLAG was incubated with a radiolabeled oligonucleotide probe harboring the HtrA4-GBS or HtrA4-GBSmt sequence. As shown in the left panel of FIG. 2E, GCM1-FLAG specifically bound to HtrA4-GBS, but not HtrA4-GBSmt, and the complex of GCM1 and HtrA4-GBS was supershifted by an anti-GCM1 antibody, indicating that there is a direct interaction between GCM1 and HtrA4-GBS. Consistently, in vivo interaction between GCM1 and HtrA4-GBS was confirmed by ChIP analysis in BeWo cells (FIG. 2E, right panel). Taken together, these results suggested that HtrA4 is a bona fide GCM 1 target gene.

### Expression of HtrA4 in Human Placenta

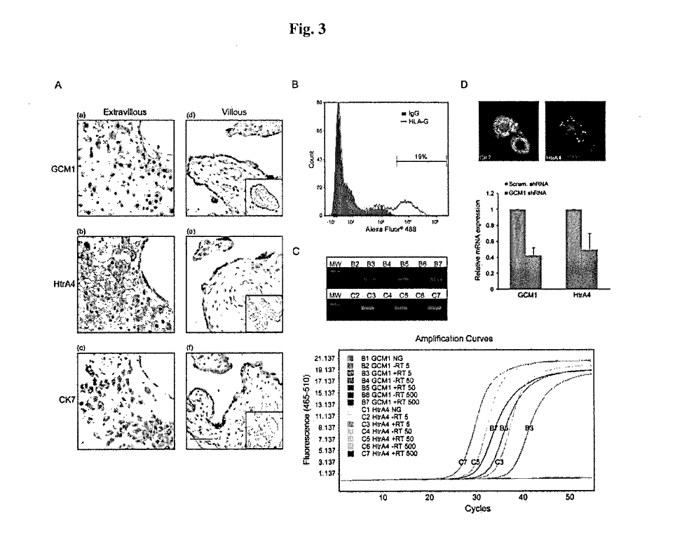

Further, the expression of GCM1 and HtrA4 in human placenta was determined by immunohistochemistry. Term placental tissue sections were immunostained using GCM1, HtrA4, and CK7 (a trophoblast marker) antibodies. While GCM1 was detected in the nuclei of both the villous syncytiotrophoblast layer and cells in the basal plate (FIG. 3A, panels a and d), HtrA4 was primarily detected in the cytoplasm of cells and the extracellular space between cells in the basal plate (FIG. 3A, compare panels b and e). Of note, both GCM1- and HtrA4-positive cell populations corresponded with the population of CK7-positive EVTs in the consecutive sections of basal plate (FIG. 3A, panels a-c).

To examine whether both GCM1 and HtrA4 are coexpressed in the interstitial EVTs of basal plate, EVTs were purified from primary trophoblast cells by flow cytometry analysis using an Ab against human leukocyte antigen-G (HLA-G), which is an EVT surface marker (27) (FIG. 3B). The levels of GCM1 and HtrA4 transcripts were analyzed in increasing number of purified EVTs (5, 50, and 500 cells) by real-time PCR analysis with SYBR green and a LightCycler instrument. As shown in the lower panel of FIG. 3C, the amplification curves of GCM1 and HtrA4 transcripts were only detected in the RNA samples converted to cDNA by reverse transcriptase (RT). Importantly, the amplification cycle numbers of initial detection of both GCM1 and HtrA4 transcripts were inversely proportional to the input cell numbers. The PCR-amplified GCM1 and HtrA4 fragments were also analyzed by gel electrophoresis and were only detected in the reverse-transcribed samples (FIG. 3C, upper panel). Immunofluorescence microscopy was performed on the purified EVTs with CK7, GCM1, and HtrA4 Abs. As shown in the upper panel of FIG. 3D, confocal analysis revealed that GCM1 and HtrA4 are colocalized in CK7-positive EVTs. Moreover, the HtrA4 transcript level was decreased in the purified EVTs when GCM1 was knocked down (FIG. 3D, lower panel). Taken together, these results indicate that GCM1 regulates HtrA4 expression in the EVTs of basal plate of human placenta.

Regulation of placental cell invasion by HtrA4

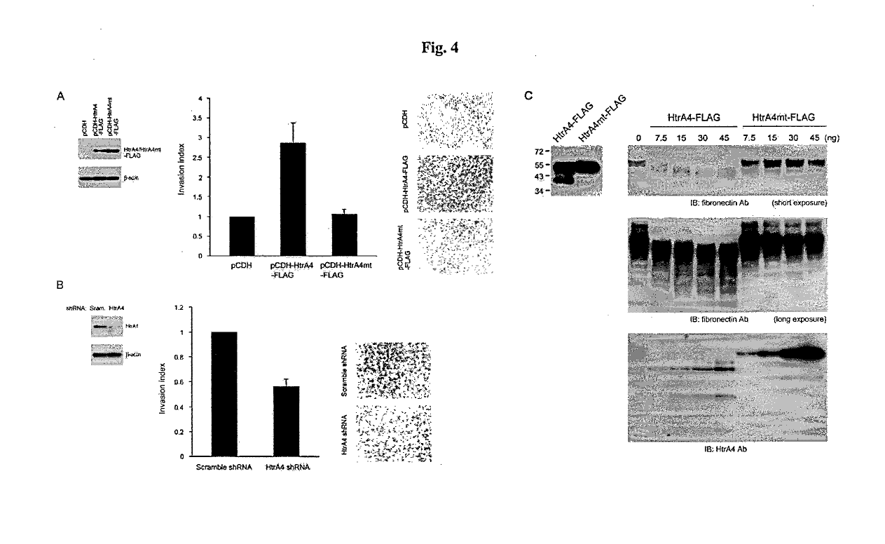

The regulatory activity of HtrA4 on placental cell invasion was investigated. Because the endogenous level of HtrA4 protein is low in JAR cells and high in BeWo cells (FIGS. 2A and B), JAR and BeWo cells stably expressing HtrA4-FLAG and HtrA4 shRNA, respectively, were established for use in the invasion assays. As compared with the mock JAR cells, the invasion activity was enhanced in JAR cells expressing the wild-type HtrA4-FLAG, but not in the cells expressing the protease-dead mutant HtrA4mt-FLAG (FIG. 4A). Consistently, knockdown of HtrA4 in BeWo cells suppressed the invasion activity of BeWo cells (FIG. 4B). This may due to the activity of HtrA4 in cleaving extracellular matrix proteins to promote cell invasion.

Wild-type HtrA4-FLAG and HtrA4mt-FLAG proteins were purified from the culture media of 293T cells transfected with pHtrA4-FLAG or pHtrA4mt-FLAG. The purified HtrA4-FLAG and HtrA4mt-FLAG proteins were incubated with recombinant fibronectin, followed by immunoblotting with anti-fibronectin Ab. As shown in FIG. 4C, cleavage of fibronectin by HtrA4-FLAG, but not HtrA4mt-FLAG, was observed in a dose-dependent manner. These results indicate that HtrA4 may facilitate cell invasion through digestion of the ECM protein fibronectin and affect the interaction between fibronectin and its integrin receptor, which is critical for placental cell invasion (14).

Decreased expression of HtrA4 by hypoxia and in preeclamptic placentas

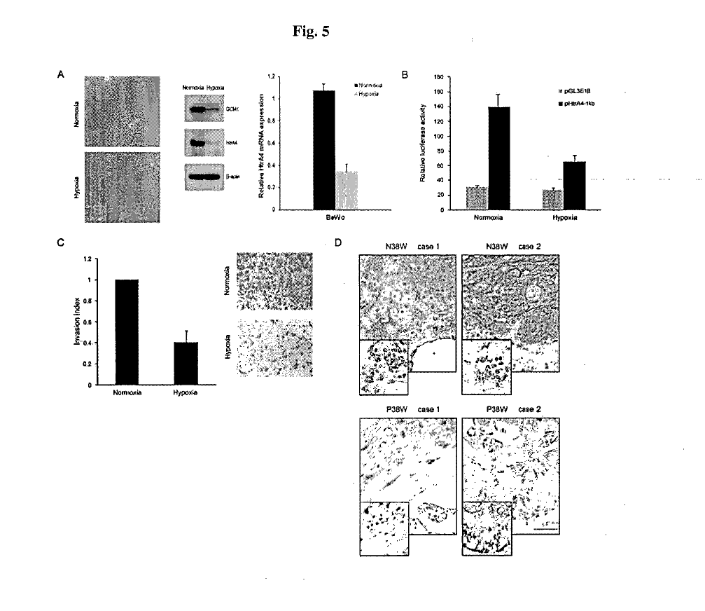

Since the GCM 1 activity is downregulated by hypoxia and in preeclampsia (10, 12), the levels of HtrA4 protein and transcript were measured in BeWo cells exposed to normoxic and hypoxic conditions to explore the correlation between HtrA4 and preeclampsia. The morphology of BeWo cells under normoxic and hypoxic conditions was not significantly different (FIG. 5A). The GCM1 protein level was decreased in the hypoxic BeWo cells. Importantly, both the HtrA4 protein and transcript levels were also decreased in the hypoxic BeWo cells (FIG. 5A). Moreover, the promoter activity of HtrA4 was suppressed by hypoxia as the luciferase activity directed by pHtrA4-1 kb was significantly decreased in BeWo cells transfected with pHtrA4-1 kb and incubated under hypoxic conditions (FIG. 5B). Accordingly, the invasion activity of BeWo cells was significantly impaired under hypoxia (FIG. 5C). Because preeclampsia is associated with placental hypoxia, it was demonstrated by immunohistochemistry that HtrA4 expression was significantly lower in preeclamptic placentas as compared with the gestation age-matched normal placentas (FIG. 5D). These results indicate that trophoblast invasion impaired by hypoxia or in preeclampsia may be attributed to the suppression of HtrA4 expression caused by decreased GCM1 activity.

### HtrA4 Suppresses Cell-Cell Fusion Mediated by Syncytin-1

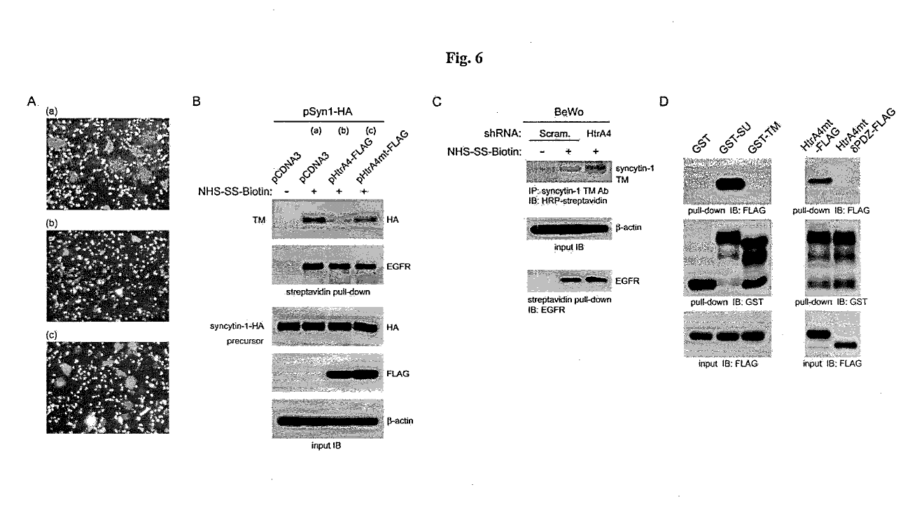

Although GCM1 regulates syncytin-1 expression to mediate cell-cell fusion for syncytiotrophoblast formation, cell-cell fusion of EVTs does not occur given that syncytin-1 is expressed in these cells. To reconcile this discrepancy, whether HtrA4 affects the cell-cell fusion mediated by syncytin-1 was investigated. To this end, a co-culture system was established to monitor cell-cell fusion by plating in the same cell culture dish a population of 293T cells expressing syncytin-1-HA and a second population of 293T cells co-expressing enhanced green fluorescent protein (EGFP) plus empty vector or EGFP plus HtrA4-FLAG. Syncytia were detected in the co-culture of syncytin-1-HA-expressing 293T cells with 293T cells co-expressing EGFP plus empty vector (panel (A) in FIG. 6A). Interestingly, syncytium formation was significantly decreased when syncytin-1-HA-expressing 293T cells were co-cultured with 293T cells co-expressing EGFP plus HtrA4-FLAG (panel b in FIG. 6A), suggesting that HtrA4 suppresses syncytin-1-HA-mediated cell-cell fusion. Moreover, this suppressive effect of HtrA4 depended on its protease activity as expression of HtrA4mt-FLAG did not significantly affect cell-cell fusion mediated by syncytin-1-HA (panel c in FIG. 6A).

How HtrA4 suppresses syncytin-1 activity was further investigated by analysis of the protein level of syncytin-1 on cell surfaces. The 293T cells of the abovementioned co-culture studies were subjected to biotinylation, followed by pull-down assay with streptavidin-conjugated matrix and HA monoclonal antibody (mAb). As shown in FIG. 6B, the protein level of the TM subunit of syncytin-1-HA decreased in the presence of HtrA4-FLAG, but not HtrA4mt-FLAG. The observed negative effect of HtrA4 was specific to surface-bound syncytin-1-HA as the level of surface-bound EGFR was not affected (FIG. 6B). The level of surface-bound syncytin-1 in BeWo cells expressing scramble or HtrA4 shRNA was also examined. To this end, an antibody recognizing the TM subunit of syncytin-1 was used to precipitate the biotinylated TM subunit of syncytin-1 on cell surfaces. Indeed, knockdown of HtrA4 significantly increased the protein level of syncytin-1 TM subunit on the surface of BeWo cells (FIG. 6C). Therefore, HtrA4 may cleave syncytin-1 to impair its fusogenic activity.

Further, the role of SU and TM subunits of syncytin-1 in interaction with HtrA4 was analyzed by pull-down analysis, using HtrA4mt-FLAG as the prey to prevent proteolysis of the bait proteins, GST-SU and GST-TM, which are GST fusion proteins harboring the SU and TM subunits, respectively. As shown in the left panel of FIG. 6D, HtrA4mt-FLAG was pulled down by GST-SU, but not GST-TM, indicating that HtrA4 is capable of binding to the SU domain.

Moreover, the role of the PDZ domain in HtrA4 for interaction with the SU subunit of syncytin-1 was examined. HtrA4mt-FLAG or its PDZ deletion variant, HtrA4mt6PDZ-FLAG was incubated with GST-SU in pull-down analysis. Interestingly, deletion of PDZ domain significantly decreased the interaction between HtrA4 and the SU subunit of syncytin-1 (FIG. 6D, right panel). Taken together, these results suggested that HtrA4 recognizes the SU subunit of syncytin-1 through its PDZ domain.

### Discussions

It has been demonstrated that the placental transcription factor GCM1 regulates placental cell invasion through transactivation of HtrA4 serine protease. Further, it was found that HtrA4 may cleave the ECM fibronectin to facilitate placental cell invasion. Collectively, these findings revealed a novel regulatory mechanism of EVT invasion by GCM1 and HtrA4.

Members of HtrA family are involved in different biological functions. HtrA2 is a mitochondrial serine protease that promotes apoptosis via interaction with and proteolysis of the IAP proteins, which contributes to caspase activation (13). Expression of both HtrA1 and -3 are highly detected in decidual cells and villous trophoblasts, but barely in interstitial EVTs (21, 31). In contrast, it is demonstrated here that HtrA4 is highly expressed in the interstitial EVTs, which may be attributed to a functional GCM1 responsive element in the proximal region of HtrA4 promoter. Interestingly, HtrA1 and -3 have been reported to inhibit trophoblast migration and invasion and the protease activity of HtrA1 was shown to be dispensable in the process (1, 34).

The results obtained from this study show that HtrA4 is a downstream effector of GCM1 stimulating placental cell invasion most likely through proteolysis of ECM proteins. Clinically, elevated expression of both HtrA1 and -3 is detected in preeclampsia (1, 21). Moreover, HtrA3 expression is enhanced under hypoxic conditions (21). Here, it was shown that HtrA4 expression is suppressed in BeWo cells by hypoxia, and so too the invasiveness of BeWo cells. Further, it was shown that HtrA4 expression decreases in preeclampsia, indicating that the level of this protein can serve as a indicia for preeclampsia development. As placental hypoxia is associated with preeclampsia and enhances GCM1 degradation, the present study suggested that HtrA4 expression is compromised by decreased GCM1 activity, which may contribute to the incomplete trophoblast invasion commonly found in preeclamptic placentas.

Immunohistochemistry indicated that HtrA4 is primarily expressed in interstitial EVTs, but not villous trophoblasts. Because GCM1 is expressed in both types of cells, it is suggested that a cell/promoter context-dependent mechanism may be involved to favor HtrA4 expression in EVTs over villous trophoblasts. Moreover, expression of GCM1 target genes in the two cell types may be differentiated due to the different environmental cues in the villus and uterine decidua. Interestingly, it was observed that forskolin, a cAMP stimulant, exerts a higher stimulatory effect on syncytin-1 expression than HtrA4, which correlates with the stimulatory effect of forskolin on cell-cell fusion.

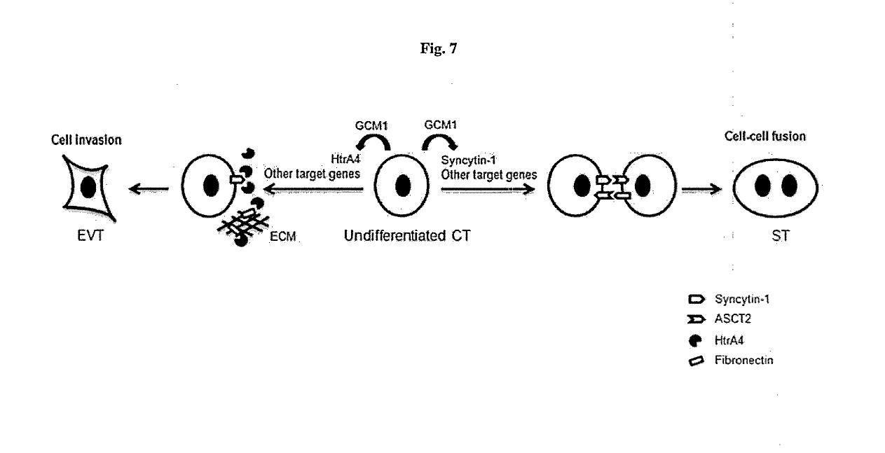

This study demonstrated that syncytin-1 is a substrate of HtrA4. In this scenario, the PDZ domain of HtrA4 is crucial for recognition of the SU subunit of syncytin-1. As a result, HtrA4 decreases the level of surface syncytin-1 to suppress cell-cell fusion mediated by syncytin-1. These observations support an additional function of HtrA4 in the suppression of EVT fusion and may reconcile the above described paradox (FIG. 7).

The results from this study also showed that the PDZ domain of HtrA4 interacts with the SU subunit of syncytin-1, suggesting that the protease activity of HtrA4 may be activated by this interaction to facilitate the proteolysis of surface-bound syncytin-1.

### REFERENCES

1. Ajayi, F., N. Kongoasa, T. Gaffey, Y. W. Asmann, W. J. Watson, A. Bald, P. Lala, V. Shridhar, B. Brost, and J. Chien. 2008. Elevated expression of serine protease HtrA1 in preeclampsia and its role in trophoblast cell migration and invasion. Am. J. Obstet. Gynecol. 199:557 e551-510.
2. Anson-Cartwright, L., K. Dawson, D. Holmyard, S. J. Fisher, R. A. Lazzarini, and J. C. Cross. 2000. The glial cells missing-1 protein is essential for branching morphogenesis in the chorioallantoic placenta. Nat. Genet. 25:311-314.
3. Baczyk, D., A. Satkunaratnam, B. Nait-Oumesmar, B. Huppertz, J. C. Cross, and J. C. Kingdom. 2004. Complex patterns of GCM1 mRNA and protein in villous and extravillous trophoblast cells of the human placenta. Placenta 25:553-559.
4. Blaise, S., N. de Parseval, L. Benit, and T. Heidmann. 2003. Genomewide screening for fusogenic human endogenous retrovirus envelopes identifies syncytin 2, a gene conserved on primate evolution. Proc. Natl. Acad. Sci. U.S.A. 100:13013-13018.
5. Blond, J. L., D. Lavillette, V. Cheynet, O. Bouton, G. Oriol, S. Chapel-Fernandes, B. Mandrand, F. Mallet, and F. L. Cosset. 2000. An envelope glycoprotein of the human endogenous retrovirus HERV-W is expressed in the human placenta and fuses cells expressing the type D mammalian retrovirus receptor. J. Virol. 74:3321-3329.
6. Cartwright, J. E., R. Fraser, K. Leslie, A. E. Wallace, and J. L. James. 2010. Remodelling at the maternal-fetal interface: relevance to human pregnancy disorders. Reproduction 140:803-813.
7. Chang, C., P. T. Chen, G. D. Chang, C. J. Huang, and H. Chen. 2004. Functional characterization of the placental fusogenic membrane protein syncytin. Biol. Reprod. 71:1956-1962.
8. Chang, C. W., H. C. Chuang, C. Yu, T. P. Yao, and H. Chen. 2005. Stimulation of GCMa transcriptional activity by cyclic AMP/protein kinase A signaling is attributed to CBP-mediated acetylation of GCMa. Mol. Cell. Biol. 25:8401-8414.
9. Chang, M., D. Mukherjea, R. M. Gobble, K. A. Groesch, R. J. Tony, and D. S. Tony. 2008. Glial cell missing 1 regulates placental growth factor (PGF) gene transcription in human trophoblast. Biol. Reprod. 78:841-851.
10. Chen, C. P., C. Y. Chen, Y. C. Yang, T. H. Su, and H. Chen. 2004. Decreased placental GCM1 (glial cells missing) gene expression in pre-eclampsia. Placenta 25:413-421.
11. Chen, C. P., L. F. Chen, S. R. Yang, C. Y. Chen, C. C. Ko, G. D. Chang, and H. Chen. 2008. Functional characterization of the human placental fusogenic membrane protein syncytin 2. Biol. Reprod. 79:815-823.
12. Chiang, M. H., F. Y. Liang, C. P. Chen, C. W. Chang, M. L. Cheong, L. J. Wang, C. Y. Liang, F. Y. Lin, C. C. Chou, and H. Chen. 2009. Mechanism of hypoxia-induced GCM1 degradation: implications for the pathogenesis of preeclampsia. J. Biol. Chem. 284:17411-17419.
13. Clausen, T., C. Southan, and M. Ehrmann. 2002. The HtrA family of proteases: implications for protein composition and cell fate. Mol. Cell. 10:443-455.
14. Damsky, C. H., C. Librach, K. H. Lim, M. L. Fitzgerald, M. T. McMaster, M. Janatpour, Y. Zhou, S. K. Logan, and S. J. Fisher. 1994. Integrin switching regulates normal trophoblast invasion. Development 120:3657-3666.
15. Drewlo, S., M. Czikk, D. Baczyk, S. Lye, and J. Kingdom. 2011. Glial cell missing-1 mediates over-expression of tissue inhibitor of metalloproteinase-4 in severe pre-eclamptic placental villi. Hum. Reprod. 26:1025-1034.
16. Fitzgerald, J. S., S. A. Tsareva, T. G. Poehlmann, L. Berod, A. Meissner, F. M. Corvinus, B. Wiederanders, E. Pfitzner, U. R. Markert, and K. Friedrich. 2005. Leukemia inhibitory factor triggers activation of signal transducer and activator of transcription 3, proliferation, invasiveness, and altered protease expression in choriocarcinoma cells. Int. J. Biochem. Cell Biol. 37:2284-2296.
17. Holtan, S. G., D. J. Creedon, P. Haluska, and S, N. Markovic. 2009. Maycer and pregnancy: parallels in growth, invasion, and immune modulation and implications for maycer therapeutic agents. Mayo Clin. Proc. 84:985-1000.
18. Hung, A. Y., and M. Sheng. 2002. PDZ domains: structural modules for protein complex assembly. J. Biol. Chem. 277:5699-5702.
19. Knerr, I., S. W. Schubert, C. Wich, K. Amann, T. Aigner, T. Vogler, R. Jung, J. Dotsch, W. Rascher, and S. Hashemolhosseini. 2005. Stimulation of GCMa and syncytin via cAMP mediated PKA signaling in human trophoblastic cells under normoxic and hypoxic conditions. FEBS Lett. 579:3991-3998.
20. Knofler, M., and J. Pollheimer. 2012. IFPA Award in Placentology Lecture: Molecular regulation of human trophoblast invasion. Placenta 33 Suppl:S55-62.
21. Li, Y., M. Puryer, E. Lin, K. Hale, L. A. Salamonsen, U. Manuelpillai, S. Tong, W. Chan, E. M. Wallace, and G. Nie. 2011. Placental HtrA3 is regulated by oxygen tension and serum levels are altered during early pregnancy in women destined to develop preeclampsia. J. Clin. Endocrinol. Metab. 96:403-411.
22. Liang, C. Y., L. J. Wang, C. P. Chen, L. F. Chen, Y. H. Chen, and H. Chen. 2010. GCM1 regulation of the expression of syncytin 2 and its cognate receptor MFSD2A in human placenta. Biol. Reprod. 83:387-395.
23. Lin, F. Y., C. W. Chang, M. L. Cheong, H. C. Chen, D. Y. Lee, G. D. Chang, and H. Chen. 2011. Dual-specificity phosphatase 23 mediates GCM1 dephosphorylation and activation. Nucleic Acids Res. 39:848-861.
24. Malassine, A., S. Blaise, K. Handschuh, H. Lalucque, A. Dupressoir, D. Evain-Brion, and T. Heidmann. 2007. Expression of the fusogenic HERV-FRD Env glycoprotein (syncytin 2) in human placenta is restricted to villous cytotrophoblastic cells. Placenta 28:185-191.
25. Malassine, A., K. Handschuh, V. Tsatsaris, P. Gerbaud, V. Cheynet, G. Oriol, F. Mallet, and D. Evain-Brion. 2005. Expression of HERV-W Env glycoprotein (syncytin) in the extravillous trophoblast of first trimester human placenta. Placenta 26:556-562.
26. Martins, L. M., B. E. Turk, V. Cowling, A. Borg, E. T. Jarrell, L. C. Maytley, and J. Downward. 2003. Binding specificity and regulation of the serine protease and PDZ domains of HtrA2/Omi. J. Biol. Chem. 278:49417-49427.
27. McMaster, M. T., C. L. Librach, Y. Zhou, K. H. Lim, M. J. Janatpour, R. DeMars, S. Kovats, C. Damsky, and S. J. Fisher. 1995. Human placental HLA-G expression is restricted to differentiated cytotrophoblasts. J. Immunol. 154:3771-3778.
28. Mi, S., X. Lee, X. Li, G. M. Veldman, H. Finnerty, L. Racie, E. LaVallie, X. Y. Tang, P. Edouard, S. Howes, J. C. Keith, Jr., and J. M. McCoy. 2000. Syncytin is a captive retroviral envelope protein involved in human placental morphogenesis. Nature 403:785-789.
29. Muir, A., A. M. Lever, and A. Moffett. 2006. Human endogenous retrovirus-W envelope (syncytin) is expressed in both villous and extravillous trophoblast populations. J. Gen. Virol. 87:2067-2071.
30. Murwantoko, M. Yano, Y. Ueta, A. Murasaki, H. Kanda, C. Oka, and M. Kawaichi. 2004. Binding of proteins to the PDZ domain regulates proteolytic activity of HtrA1 serine protease. Biochem. J. 381:895-904.
31. Nie, G., K. Hale, Y. Li, U. Manuelpillai, E. M. Wallace, and L. A. Salamonsen. 2006. Distinct expression and localization of serine protease HtrA1 in human endometrium and first-trimester placenta. Deva Dyn. 235:3448-3455.
32. Runyon, S. T., Y. Zhang, B. A. Appleton, S. L. Sazinsky, P. Wu, B. Pan, C. Wiesmann, N.J. Skelton, and S. S. Sidhu. 2007. Structural and functional analysis of the PDZ domains of human HtrA1 and HtrA3. Protein Sci. 16:2454-2471.
33. Schreiber, J., E. Riethmacher-Sonnenberg, D. Riethmacher, E. E. Tuerk, J. Enderich, M. R. Bosl, and M. Wegner. 2000. Placental failure in mice lacking the mammalian homolog of glial cells missing, GCMa. Mol. Cell. Biol. 20:2466-2474.
34. Singh, H., S. I. Makino, Y. Endo, and G. Nie. 2010. Inhibition of HTRA3 stimulates trophoblast invasion during human placental development. Placenta 31:1085-1092.
35. Xie, T. X., D. Wei, M. Liu, A. C. Gao, F. Ali-Osman, R. Sawaya, and S. Huang. 2004. Stat3 activation regulates the expression of matrix metalloproteinase-2 and tumor invasion and metastasis. Oncogene 23:3550-3560.
36. Yang, C. S., C. Yu, H. C. Chuang, C. W. Chang, G. D. Chang, T. P. Yao, and H. Chen. 2005. FBW2 targets GCMa to the ubiquitin-proteasome degradation system. J. Biol. Chem. 280:10083-10090.
37. Yu, C., K. Shen, M. Lin, P. Chen, C. Lin, G. D. Chang, and H. Chen. 2002. GCMa regulates the syncytin-mediated trophoblastic fusion. J. Biol. Chem. 277:50062-50068

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art may easily ascertain the essential characteristics of the present invention, and without departing from the spirit and scope thereof, may make various changes and modifications of the invention to adapt it to various usages and conditions. Thus, other embodiments are also within the claims.

What is claimed is:

1. A method of diagnosing a pregnancy disorder in a subject, the method comprising:
providing a biological sample of a pregnant subject;
measuring a level of High Temperature Requirement A4 (HtrA4) in the biological sample; and
identifying the pregnant subject as having or at risk for a pregnancy disorder based on the level of HtrA4,
wherein the pregnant subject is identified as a subject having or at risk for the pregnancy disorder if the level of HtrA4 deviates from a reference value.

2. The method of claim 1, wherein the pregnancy disorder is preeclampsia or intra-uterine growth retardation (IUGR).

3. The method of claim 1, wherein the biological sample is a serum sample or a plasma sample.

4. The method of claim 1, wherein the biological sample is obtained from the pregnant subject at 10 weeks to 38 weeks of pregnancy.

5. The method of claim 1, wherein the level of HtrA4 is a level of HtrA4 protein or a HtrA4 gene transcript.

6. The method of claim 5, wherein the level of HtrA4 protein is measured by an assay involving an anti-HtrA4 antibody or an assay involving a nucleic acid complementary to the HtrA4 gene transcript or a fragment thereof.

7. The method of claim 6, wherein the assay is an enzyme-linked immunosorbent assay (ELISA), an immunoblotting assay, a hybridization assay, or a real-time PCR assay.

8. The method of claim 1, further comprising monitoring progress of the pregnancy disorder in the pregnant subject or monitoring the health of the baby if the subject is identified as having or at risk for the pregnancy disorder.

9. A method of monitoring development of a pregnancy disorder in a subject, the method comprising:
providing a first biological sample of a pregnant subject at a first gestation stage;
measuring a first level of High Temperature Requirement A4 (HtrA4) in the first biological sample;
providing a second biological sample of the pregnant subject at a second gestation stage subsequent to the first gestation stage;
measuring a second level of HtrA4 in the second biological sample; and
assessing development of a pregnancy disorder in the pregnant subject based on the change of the levels of HtrA4 in the first and second biological samples,
wherein the second level of HtrA4 deviating from the first level HtrA4 indicates that the pregnancy disorder progresses in the pregnant subject or the pregnant subject has developed or is at risk for developing the pregnancy disorder.

10. The method of claim 9, wherein the pregnancy disorder is preeclampsia or intra-uterine growth retardation (IUGR).

11. The method of claim 9, wherein the first and second biological samples are serum samples or plasma samples.

12. The method of claim 9, wherein the first biological sample is obtained from the pregnant subject at 10 weeks to 38 weeks of pregnancy.

13. The method of claim 9, wherein the second biological sample is obtained from the pregnant subject at least 2 weeks after obtaining the first biological sample.

14. The method of claim 9, wherein the level of HtrA4 is a level of HtrA4 protein or a level of HtrA4 gene transcript.

15. The method of claim 9, wherein the level of HtrA4 protein is measured by an assay involving an anti-HtrA4 antibody or an assay involving a nucleic acid complementary to the HtrA4 gene transcript or a fragment thereof.

16. The method of claim 15 wherein the assay is an enzyme-linked immunosorbent assay (ELISA), an immunoblotting assay, a hybridization assay, or a real-time PCR.

17. The method of claim 9, wherein the first and the second biological samples are obtained before and after a treatment of the pregnancy disorder or during the course of the treatment.
